(19) United States
(12) Patent Application Publication
(10) Pub. No.: US 2013/0115207 A1
(43) Pub. Date: May 9, 2013

(54) METHODS FOR THE TREATMENT OF AUTOIMMUNE DISEASES

(71) Applicant: Denise L. Faustman, Boston, MA (US)

(72) Inventor: Denise L. Faustman, Boston, MA (US)

(73) Assignee: The General Hospital Corporation, Boston, MA (US)

(21) Appl. No.: 13/639,780

(22) PCT Filed: Apr. 7, 2011
(86) PCT No.: PCT/US11/31601

§ 371 Date: Jan. 18, 2013

Related U.S. Application Data
(60) Provisional application No. 61/322,695, filed on Apr. 9, 2010.

(51) Int. Cl. A61K 35/76

(52) U.S. Cl. CPC A61K 35/763
USPC 424/133.1; 424/281.1

(57) ABSTRACT

The invention provides methods of treating a mammal (e.g., a human) having or at risk of having an autoimmune disease by administering a composition that includes all or a portion of a viral polypeptide or a nucleic acid encoding a viral peptide (e.g., a live, killed, attenuated, or inactivated virus) or a composition that includes an immunosuppressive agent (e.g., an anti-CD3 antibody), and compositions for use in treating an autoimmune disease in the mammal.

### FIELD OF THE INVENTION

In general, the present invention relates to the treatment of autoimmune disorders.

### BACKGROUND OF THE INVENTION

Tumor necrosis factor-alpha (TNF-α) is a naturally occurring cytokine that was described in 1975 as the serum factor induced after Bacillus Calmette-Guérin (BCG) injection, an avirulent form of tuberculosis, as a means to fight tumors (Carswell et al., Proc. Natl. Acad. Sci. U.S.A. 72:3666-3670, 1975). The cloning of TNF-α and its two receptors uncovered sequence homology to the genomes of microbial pathogens (e.g., Loetscher et al., Cell 62:351, 1990). This surprising sequence overlap represents a system of intricate microbial responses to modulate host TNF-α secretion and the activity of its receptors (Rahman et al., PloS Pathogens 2:66, 2006).

TNF-α expression is induced by diverse bacteria, parasites, and viruses as a host first line defense to infections. Viruses, such as the Epstein-Barr virus, encode receptors and proteins that even augment TNF-α and TNF-α signaling (Liebowitz, New Engl. J. Med. 338:1461-1463, 1998; Guasparri et al., Blood 111:3813-3821, 2008; Wang et al., Cell 43:831-840, 1985). Alternatively, a variety of viruses have been shown to express proteins that repress TNF-α signaling activity and function in the host (Rahman et al., PloS Pathogens 2:66, 2006). Some evidence suggests that viral infections (e.g., Epstein-Barr virus infections) may cause autoimmune disease (Sairenji et al., Diabetologia 34:33-39, 1991).

Autoimmune diseases are believed to involve immune responses to the body's own components that are not observed under normal conditions, which result in a pathological state that causes various tissue disorders and/or functional disorders. Autoimmune diseases are broadly classified into systemic autoimmune diseases and organ-specific autoimmune diseases according to their characteristics. Typical examples of autoimmune diseases include insulin-dependent diabetes (also known as type 1 diabetes), systemic lupus erythematosus, chronic rheumatoid arthritis, Hashimoto's disease, alopecia areata, ankylosing spondylitis, antiphospholipid syndrome, autoimmune Addison's disease, autoimmune hemolytic anemia, autoimmune hepatitis, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatricial pemphigoid, CREST syndrome, cold agglutinin disease, Crohn's disease, discoid lupus, ulcerative colitis, psoriatic arthritis, essential mixed cryoglobulinemia, fibromyalgia-fibromyositis, Graves' disease, Guillain-Barré, hypothyroidism, idiopathic pulmonary fibrosis, idiopathic thrombocytopenia purpura (ITP), IgA nephropathy, juvenile arthritis, lichen planus, lupus, Ménière's disease, mixed connective tissue disease, multiple sclerosis, myasthenia gravis, pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjögren's syndrome, Stiff-Man syndrome, Devic's disease, Takayasu arteritis, temporal arteritis/giant cell arteritis, ulcerative colitis, uveitis, vasculitis, vitiligo, and Wegener's granulomatosis.

There remains a need for methods and compositions for treating autoimmune diseases.

### SUMMARY OF THE INVENTION

In a first aspect, the invention provides methods of treating a mammal (e.g., a human) having an autoimmune disease by administering to the mammal a composition containing a viral polypeptide or a nucleic acid molecule encoding the viral polypeptide. In another embodiment of the methods of the invention, the composition administered to the mammal may contain a live, killed, attenuated, or inactivated virus containing the viral polypeptide or nucleic acid molecule encoding the viral polypeptide. In an embodiment, the virus is attenuated or inactivated.

In different embodiments of the methods of the invention, the live, killed, attenuated, and/or inactivated virus is selected from the group of poxvirus, vaccinia virus, tanapox virus, herpes virus, Epstein Barr Virus (EBV), cytomegalovirus, herpesvirus Saimiri (HVS), hepatitis B virus, parvovirus H1, human immunodeficiency virus, hepatitis C virus, influenza virus, respiratory syncytial virus (RSV), measles virus, vesicular stomalitis virus, dengue virus, and ebola virus. In an embodiment, the virus is EBV. In additional embodiments of the above methods, the virus is selected from poxyiridae virus with deletion or inactivation of T2 proteins or inactivation of viroreceptors, vaccinia virus with deletion or inactivation of the BBR gene, Tanapox virus with deletion or inactivation of the 38 kDa protein, herpes simplex virus with inactivation of caspase 3-blockage or HVEM, inactive HVS, virulent parvovirus H1, inactivated HIV, hepatitis C virus with deletion or inactivation of the core protein or NS5A, inactive influenza virus, RSV with deletion or mutation of cysteines, inactive measles virus, inactive vesicular stomalitis virus, inactive dengue virus, or inactive ebola virus.

In additional embodiments of the methods of the invention, the viral polypeptide includes all or a portion of a naturally occurring viral polypeptide. In further embodiments of the invention, the composition contains two or more viral polypeptides or nucleic acid molecules encoding the two or more viral polypeptides. In another embodiment of the invention, the viral polypeptide is an EBV polypeptide LMP1, Herpesvirus Saimiri STP protein, Hepatitis B Virus HBx protein, human immunodeficiency virus Tat protein, Hepatitis C Virus core protein, influenza virus hemagglutinin protein, an antagonist of T2 poxyiridae protein, an antagonist of vaccinia BBR protein, an antagonist of Tanapox virus 38 kDa protein, an antagonist of Herpes simplex virus HVEM protein, an antagonist of Hepatitis C Virus core protein, or an antagonist that binds to the death domain of TNFR1. In any of the above methods, the viral polypeptide may further include a non-virus-derived polypeptide. In addition, in any of the above methods, the composition may induce expression of tumor necrosis factor-alpha (TNF-α) in the mammal, agonize a TNF-α receptor in an autoreactive immune cell of the mammal, and/or induce activation of the NF-kappa B pathway in an autoreactive immune cell of the mammal. In any of the embodiments described herein, the mammal is a human.

In any of the above methods of the invention, the autoimmune disease is selected from alopecia areata, ankylosing spondylitis, antiphospholipid syndrome, autoimmune Addison's disease, autoimmune hemolytic anemia, autoimmune hepatitis, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatricial pemphigoid, CREST syndrome, cold agglutinin disease, Crohn's disease, discoid lupus, systemic lupus erythmatosous, ulcerative colitis, psoriatic arthritis, essential mixed cryoglobulinemia, fibromyalgia-fibromyositis, Graves' disease, Guillain-Barré, Hashimoto's thyroiditis, hypothyroidism, idiopathic pulmonary fibrosis, idiopathic thrombocytopenia purpura (ITP), IgA nephropathy, insulin-dependent diabetes, juvenile arthritis, lichen planus, lupus, Ménière's disease, mixed connective tissue disease, multiple sclerosis, myasthenia gravis, pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjögren's syndrome, Stiff-Man syndrome, Devic's disease, Takayasu arteritis, temporal arteritis/giant cell arteritis, ulcerative colitis, uveitis, vasculitis, vitiligo, and Wegener's granulomatosis. In particular, the autoimmune disease is insulin-dependent diabetes (also referred to herein as type 1 diabetes mellitus (T1DM)).

In additional embodiments of the methods of the invention, the treatment results in at least a 1% increase in C-peptide levels in the mammal relative to the C-peptide levels in the mammal prior to the treatment. In another embodiment of the above methods, the mammal is a long-term insulin dependent diabetic.

In different embodiments of the above methods, the composition may contain a TNF-α receptor agonist (e.g., TNF-α receptor 2 agonist) and/or an intracellular mediator of the TNF-α signaling pathway. In another embodiment of the invention, the TNF-α receptor agonist is an antibody (e.g., an antibody that agonizes TNF-α receptor 2).

In additional embodiments of the methods of the invention, the composition further includes one or more TNF-α inducing substances (e.g., complete Freund's adjuvant, BCG, tissue plasminogen factor, lipopolysaccharide (LPS), interleukin-1, interleukin-2, lymphotoxin, and/or cachectin).

In a further aspect of the methods of the invention, the composition selectively kills blood cells (e.g., autoreactive CD8⁺ T cells) with increased sensitivity to cell death, and whereby killing the blood cells treats or stabilizes the autoimmune disease in the mammal (e.g., a human).

In additional embodiments of the methods of the invention, the composition is administered to the mammal (e.g., a human) prior to the development of one or more symptoms of the autoimmune disease or is administered to the mammal after the development of one or more symptoms of the autoimmune disease.

In further embodiments of the methods of the invention, the treatment: induces at least a 1% increase in autoreactive T cell death in the mammal (e.g., a human) relative to the level of autoreactive T cell death observed in the mammal prior to the treatment; induces at least a 1% increase in the number of regulatory T cells in the mammal relative to the number of regulator T cells present in the mammal prior to the treatment; induces at least a 1% decrease in autoantibody levels in the mammal relative to the autoantibody levels (e.g., anti-glutamic acid dehydrogenase (anti-GAD) or anti-pancreatic beta cell-specific zinc transporter (anti-ZnT8A) antibodies) in the mammal prior to the treatment; and/or results in a decrease in one or more symptoms of the autoimmune disease (e.g., increased levels of autoantibodies, increased levels of autoreactive T cells, loss of targeted cells (e.g., pancreatic (3-islet cells), fatigue, depression, sensitivity to cold, weight gain, muscle weakness, constipation, insomnia, irritability, weight loss, bulging eyes, muscle tremors, skin rashes, painful or swollen joints, sensitivity to the sun, loss of coordination, and paralysis).

In any of the above methods of the invention, the composition may be administered parenterally, topically, intravenously, intra-arterially, intracranially, intradermally, subcutaneously, intramuscularly, intraorbitally, intraventricularly, intraspinally, intraperitoneally, intranasally, or orally, and/or administered in one or more doses.

In additional embodiments of any of the above methods, the one or more doses of the composition are administered twice daily, daily, weekly, biweekly, monthly, bi-annually, tri-annually, quarterly, or yearly.

In a second aspect, the invention provides methods of treating a mammal (e.g., a human) having an autoimmune disease by administering to the mammal a composition that includes a live, killed, attenuated, or inactivated Epstein Barr Virus (EBV).

In a third aspect, the invention provides a method of treating a mammal (e.g., a human) having an autoimmune disease by administering a composition (e.g., an immunosuppressive agent) that reactivates a latent Epstein Barr Virus infection in the mammal. In an embodiment, the method further includes testing the mammal for the presence of EBV (e.g., due to a previous infection) prior to administering the composition (e.g., an immunosuppressive agent). In additional embodiments of this aspect of the invention, the immunosuppressive agent may be selected from cyclosporine (e.g., cyclosporin A, such as NEORAL®, SANDIMMUNE®, and SANGCYA®), an azathioprine (e.g. IMURAN®), FK-506, 15-deoxyspergualin, or an antibody (e.g., a monoclonal antibody, such as basiliximab, daclizumab, and muromonab-CD3, or a globulin, such as anti-lymphocyte globulin, anti-thymocyte globulin, and the like). In yet other embodiments, the immunosuppressive therapy is an anti-CD3 antibody (e.g., teplizumab or otelixizumab). In further embodiments of the third aspect of the invention, reactivation of the latent EBV infection may be determined using an antibody-based assay, a PCR-based assay, or by other serological methods known in the art (e.g., those described herein).

In another embodiment of the third aspect of the invention, the mammal (e.g., a human) having a latent EBV infection is administered at least one dose (e.g., 2, 3, 4, 5, 6, 7, 8, 9, or 10 doses or up to 20 or more doses) of an anti-CD3 antibody (e.g., a dose in the range of 10 μg/m2 to about 3 mg/m2). For example, a mammal having a latent EBV infection is administered an anti-CD3 antibody (e.g., teplizumab or otelixizumab) as follows: day 0 at 10 μg/m2 to 150 μg/m2; day 2 at 50 μg/m2 to 200 μg/m2; day 3 at 100 μg/m2 to 300 μg/m2; day 4 at 250 μg/m2 to 500 μg/m2; and days 5-20 at 600 μg/m2 to 900 μg/m2.

In other embodiments of the third aspect of the invention, the method includes, prior to administering the immunosuppressive therapy to the mammal (e.g., a human), detecting EBV in the mammal. The detecting step may be accomplished by performing, e.g., a diagnostic test on the mammal to determine whether the mammal has been infected with EBV. In several embodiments, the mammal may have an acute EBV infection, a chronic EBV infection, a subclinical EBV infection, a latent EBV infection, or an EBV infection due to reactivation of a latent infection. Those mammals showing positive EBV infection (whether acute, chronic, subclinical, latent, or due to reactivation) are further treated according to the methods of the invention. In several embodiments, the method includes detecting tetramer-positive T cells against EBV cells, EBV-specific antibodies (e.g., viral capsid antigen (VCA)-IgM, VCA-IgG, D early antigen (EA-D)-IgG, or Epstein Barr nuclear antigen-IgG), and/or EBV deoxyribonucleic acid (DNA) or messenger ribonucleic acid (mRNA) molecules.

In an embodiment, the method includes administering an anti-CD3 antibody (e.g., teplizumab and/or otelixizumab) as an immunosuppressive therapy to a mammal (e.g., a human) that has a positive EBV result. In several embodiments, the anti-CD3 antibody (e.g., teplizumab and/or otelixizumab) is administered in one or more doses (e.g., 2, 3, 4, 5, 6-10, 10-20 or more doses) in a range of from about 5 μg to about 200 mg (e.g., about 50 μg to about 100 mg; about 50 μg to about 50 mg; about 50 μg to about 1 mg; about 100 μg to about 50 mg; or about 150 μg to about 50 mg) per dose. In other embodiments, at least one dose of an anti-CD3 antibody (e.g., teplizumab and/or otelixizumab) as an immunosuppressive therapy is administered to a mammal at least once per day (e.g., 2, 3, 4, or 5 times per day or more); at least once per week; at least once every two weeks; at least once every month; at least once every two months; at least once every three months; at least once every six months; or at least once every year over a period of at least one day, one week, two weeks, three weeks, one month, two months, three months, six months, or one year or more. In other embodiments, at least one dose of an anti-CD3 antibody (e.g., teplizumab and/or otelixizumab) as an immunosuppressive therapy is administered to a mammal for at least one day, one week, one month, or one year or more (e.g., any number of days from 1 to 365); at least once per week for 1 to 52 weeks or more; at least once every two weeks for 2 to 52 weeks or more; at least once every month for 1 to 12 months or more; at least once every two months for 2 to 12 months; at least once every three months for 3-12 months; at least once every six months for 1 to 5 years; or at least once every year for 1 to 5 years.

In yet other embodiments of the third aspect of the invention, the autoimmune disease is selected from alopecia areata, ankylosing spondylitis, antiphospholipid syndrome, autoimmune Addison's disease, autoimmune hemolytic anemia, autoimmune hepatitis, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatricial pemphigoid, CREST syndrome, cold agglutinin disease, Crohn's disease, discoid lupus, systemic lupus erythmatosous, ulcerative colitis, psoriatic arthritis, essential mixed cryoglobulinemia, fibromyalgia-fibromyositis, Graves' disease, Guillain-Barré, Hashimoto's thyroiditis, hypothyroidism, idiopathic pulmonary fibrosis, idiopathic thrombocytopenia purpura (ITP), IgA nephropathy, insulin dependent diabetes, juvenile arthritis, lichen planus, lupus, Ménière's disease, mixed connective tissue disease, multiple sclerosis, myasthenia gravis, pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjögren's syndrome, Stiff-Man syndrome, Devic's disease, Takayasu arteritis, temporal arteritis/giant cell arteritis, ulcerative colitis, uveitis, vasculitis, vitiligo, and Wegener's granulomatosis (e.g., the autoimmune disease is insulin-dependent diabetes). In still other embodiments, the composition (e.g., an anti-CD3 antibody, such as, e.g., teplizumab and/or otelixizumab) is administered with a pharmaceutically acceptable carrier or excipient, or as a salt formulation.

### DEFINITIONS

By “detecting” or “detectable” with respect to the presence of an infectious agent (e.g., a virus) is meant the use of a clinical parameter indicative of an active, chronic, subclinical, or latent infection (e.g., an EBV infection). For example, clinical parameters indicative of an active, chronic, subclinical, or latent EBV infection include, e.g., an increase in EBV specific serology, PCR detection of an EBV nucleic acid molecule, CD8 lymphocytosis, EBV specific T cells, and change in liver enzymes. The EBV could be in a state of transient re-activation, sub-clinical re-activation, active, or chronic infection.

### BRIEF DESCRIPTION OF THE DRAWINGS

FIG. 1 is a list of viruses that inhibit or modulate expression of TNF-α (e.g., the expression of TNF-α protein or an mRNA encoding a TNF-α protein) or the activity of TNF-α (e.g., the activation of TNF-α receptor, a TNF-α signaling cascade, or an NF-κB signaling cascade).

FIGS. 2A-2C show the fluorescence-assisted cell sorting (FACS) data of purified CD8⁺ T cells from study subjects to identify T cells that are autoreactive to insulin. In these experiments, isolated CD8⁺ T cells from patients receiving BCG (FIG. 2A) or placebo (FIG. 2B), or demonstrating acute EBV infection (FIG. 2C) (triangles) (collected from subjects at weeks 0 (B), 1, 2, 3, 4, 5, 7, 8, 10, and 12), and from matched non-diabetic controls (diamonds) were incubated with fluorescently-labeled tetramers to HLA 0210 with an insulin beta 10-18 fragment (HLVEALYLV) and Sytox-green, washed twice in Hanks buffer with 2% heat inactivated bovine serum, and sorted using a flow cytometer. The percentage of CD8⁺ T cells that are autoreactive to insulin are shown. The area under the curve (AUC) was calculated for the increased number of insulin autoreactive T cells for each patient compared to their simultaneous matched control blood samples over the weeks of monitoring.

FIG. 3 shows the identification of viable and dead CD8⁺ T cells using flow cytometry. In these experiments, alive and dead CD8⁺ T cells are first separated by their forward and side light scatter properties (left panel), and then separated by their propidium iodide fluorescence (right panels).

FIGS. 4A-4C show the viability data of the insulin autoreactive CD8⁺ T cells purified from the peripheral blood of the subjects (BCG-treated, FIG. 4A; placebo-treated, FIG. 4B; and EBV infected, FIG. 4C). In these experiments, isolated CD8⁺ cells were stained with fluorescently-labeled tetramers to HLA 0210 with an insulin beta 10-18 fragment (HLVEALYLV) and co-stained for viability using propidium iodide. The percentage of dead versus live insulin-reactive CD8⁺ T cells for each subject is shown at weeks 0 (B), 1, 2, 3, 4, 5, 7, 8, 10, and 12.

FIGS. 5A-5B show the data for the number of Treg CD4⁴⁺ cells in subjects using FACS. In these experiments, purified CD4⁺ cells were stained with anti-CD4, anti-CD25^{bright}, and anti-Foxp3 antibodies (FIG. 5A) or anti-CD4, anti-CD25^{bright}, and anti-CD127^{low} antibodies (FIG. 5B) from the BCG-treated, placebo-treated, and EBV-infected subject(s) (weeks 0-12), and the BCG-treated and placebo-treated subjects (weeks 0-12), respectively.

FIGS. 6A-6C show the data for the levels of GAD autoantibodies in the BCG-treated (FIG. 6A), placebo-treated (FIG. 6B), and EBV-infected subject(s) (FIG. 6C) between weeks 0 and 20 of the study, as measured by immunoassay.

FIGS. 7A-7C show the data for the levels of protein tyrosine phosphatase (IA-2A) autoantibodies in the BCG-treated (FIG. 7A), placebo-treated (FIG. 7B), and EBV-infected subject(s) (FIG. 7C) between weeks 0 and 20 of the study, as measured by immunoassay.

FIGS. 8A-8C show the data for the levels of pancreatic beta cell-specific zinc transporter (ZnT8A) autoantibodies in BCG-treated (FIG. 8A), placebo-treated (FIG. 8B), and EBV-infected subject(s) (FIG. 8C) between weeks 0 and 20 of the study as measured by immunoassay.

FIGS. 9A-9C show the data for the levels of C-peptide in the BCG-treated (FIG. 9A), placebo-treated (FIG. 9B), and EBV-infected subject(s) (FIG. 9C) at time points throughout the study as measured by immunoassay.

FIG. 10 shows the levels of EBV-specific CD8⁺ T cells in the Andover subject and a control subject at weeks 6, 7, and 8 of the study using flow cytometric analysis of EBV-tetramer stained CD8⁺ T cells.

FIG. 11 shows the levels of both Early Antigen D IgG and VCA-IgM antibodies in the Andover patient (top and bottom, respectively) at weeks 0, 3, 6, and 8 of the study.

FIGS. 12A-12B depict changes in diabetes antigen specific CD8+ T cells with treatment with Teplizumab and is reproduced from Cernea et al, (Clinical Immunology 134:121, 2010; see FIG. 4). FIG. 12A shows representative staining for CD8 and tetramers from a single participant before (day 0) and 3 months after (day 90) treatment with Teplizumab. The numbers refer to the percentage tetramer+ of the CD8+ T cells. Bolded numbers are considered positive staining based on threshold values. FIG. 12B shows changes in the frequency of diabetes antigen specific T cells in individual patients treated with Teplizumab and untreated patients with T1DM (tetramers: InsA1 ▾, InsA2 ▴, Ins B ▪, PPI ♦, GAD , IGRP □, Flu ◯).

### DETAILED DESCRIPTION

The invention features methods for treating a mammal (e.g., a human) having or at risk of developing an autoimmune disease that involve the administration of a composition containing all or a portion of a viral polypeptide or a nucleic acid encoding the viral polypeptide or by administering an immunosuppressive agent (e.g., an anti-CD3 antibody, such as teplizumab and/or otelixizumab). In an embodiment, the mammal has an active, chronic, subclinical, or latent viral infection (e.g., an EBV infection).

The viral polypeptide may be derived from a natural polypeptide of a DNA or RNA virus and mediates (either directly or indirectly) an increase in the expression of TNF-α (e.g., an increase in the expression of TNF-α protein or an mRNA encoding a TNF-α protein) or the activity of TNF-α (e.g., an increase in the activation of a TNF-α receptor (e.g., TNF-α receptor 1 or 2, and preferably TNF-α receptor 2), a TNF-α signaling cascade, or an NF-κB signaling cascade) in a mammalian cell (in vitro or in vivo).

Viral polypeptides of the invention may include all or a contiguous portion of a naturally-occurring viral polypeptide. The viral polypeptide (whether as full-length or a fragment thereof) exhibits the characteristics described in the preceding paragraph (e.g., the ability to increase expression or activity of TNF-α). The viral polypeptide of the invention may have a sequence that is at least 80% identical (e.g., at least 85%, 90%, 95%, 96%, 97%, 98%, 99%, or even 100% identical) to the naturally-occurring viral polypeptide over a span of 10, 20, 50, 100, 200, 300, or more amino acids, or over the entire length of the naturally-occurring viral polypeptide. A nucleic acid sequence encoding a viral polypeptide of the invention may contain a nucleic acid sequence that encodes a polypeptide that is at least 80% identical (e.g., at least 85%, 90%, 95%, 96%, 97%, 98%, 99%, or even 100% identical) to all or a contiguous portion (e.g., 10, 20, 50, 100, 200, 300, or more amino acids, or over the entire length) of a naturally-occurring viral polypeptide.

Viral polypeptides of the invention exhibit at least 80% or more (e.g., 85%, 90%, 95%, 99%, or more) identity to a naturally-occurring viral polypeptide over a span of 10, 20, 50, 100, 200, 300, or more amino acids, or over the entire length of the naturally-occurring viral polypeptide, and/or may differ from naturally-occurring viral polypeptides by having one or more amino acid deletions and/or one or more conservative amino acid substitutions. The deletions or substitutions may or may not alter the biological activity of the viral polypeptide (e.g., the mutation may increase the biological activity of the viral polypeptide (e.g., the ability to stimulate TNF-α expression, activate TNF-α signaling pathways, activate TNF-α receptor, or activate NF-κB signaling pathways)). Viral polypeptides of the invention may also include amino acid substitutions or deletions relative to the naturally-occurring polypeptide that further stabilize or increase the half-life of the viral polypeptide. Mutations in the viral polypeptide may be made in specific domains of the viral polypeptide or may be made at amino acid positions that are conserved or at amino acid positions that are not conserved in the viral polypeptide sequence.

Non-limiting examples of polypeptides that may be included in the compositions of the invention include Epstein Barr virus polypeptide LMP1 (e.g., a polypeptide containing a sequence that is at least 80% or more identical to all or a contiguous portion (e.g., over a span of 10, 20, 50, 100, 200, 300, or more amino acids) of the sequence identified by NCBI Accession No. Q1HVB3), Herpesvirus Saimiri STP protein (e.g., a polypeptide containing a sequence that is at least 80% or more identical to all or a contiguous portion (e.g., over a span of 10, 20, 50, 100, 200, 300, or more amino acids) of the sequence identified by NCBI Accession No. P18347), Hepatitis B Virus HBx protein (e.g., a polypeptide containing a sequence that is at least 80% or more identical to all or a contiguous portion (e.g., over a span of 10, 20, 50, 100, 200, 300, or more amino acids) of the sequence identified by NCBI Accession No. CAA49453), human immunodeficiency virus Tat protein (e.g., a polypeptide containing a sequence that is at least 80% or more identical to all or a contiguous portion (e.g., over a span of 10, 20, 50, 100, 200, 300, or more amino acids) of the sequence identified by NCBI Accession No. AAF35362), Hepatitis C Virus core protein (e.g., a polypeptide containing a sequence that is at least 80% or more identical to all or a contiguous portion (e.g., over a span of 10, 20, 50, 100, 200, 300, or more amino acids) of the sequence identified by NCBI Accession No. ABI45512), influenza virus hemagglutinin protein (e.g., a polypeptide containing a sequence that is at least 80% or more identical to all or a contiguous portion (e.g., over a span of 10, 20, 50, 100, 200, 300, or more amino acids) of the sequence identified by NCBI Accession No. CAG28944), an antagonist of T2 poxyiridae protein, an antagonist of vaccinia BBR protein, an antagonist of Tanapox virus 38 kDa protein, an antagonist of Herpes simplex virus HVEM protein, an antagonist of Hepatitis C Virus core protein, and an antagonist that binds to the death domain of TNFR1. The viral polypeptides of the invention, whether as full-length or a fragment thereof, retain the ability to increase the expression or the activity of TNF-α.

A viral polypeptide containing a sequence that is at least 80% or more identical to a naturally-occurring viral polypeptide may also be truncated at its N- and/or C-terminus. For example, a naturally-occurring viral polypeptide may be truncated and its N- and/or C-terminus by at least 1 amino acid (e.g., at least 2, 3, 4, 5, 6, 7, 8, 9, 10, 15, 20, 25, 30, 35, 40, 45, 50, 55, 60, or 70 amino acids). A viral polypeptide containing a sequence that is at least 80% or more identical to a naturally-occurring polypeptide may also contain only the portion or structural domain of the viral polypeptide responsible for its biological effect (e.g., the ability to stimulate TNF-α expression, activate TNF-α signaling pathways, activate TNF-α receptor, or activate NF-κB signaling pathways). The total size of the viral polypeptide may be between 10 and 50 amino acids, between 10 and 100 amino acids, between 50 and 200 amino acids, between 50 and 300 amino acids, between 100 and 350 amino acids, between 200 and 500 amino acids, between 300 and 800 amino acids, or between 300 and 900 amino acids.

The invention also provides a viral polypeptide, or a nucleic acid encoding a viral polypeptide, that includes one or more (e.g., 1, 2, 3, or 4) non-virus-derived polypeptide sequences. For example, a polypeptide that contains a sequence that is at least 80% or more identical to the sequence of a naturally-occurring viral polypeptide or a fragment thereof may contain a peptide sequence at its N- and/or C-terminus that will further stimulate TNF-α expression, activate TNF-α signaling pathways, activate TBF-α receptor, or activate NF-κB signaling pathways (e.g., tissue plasminogen factor, interleukin-1, and interleukin-2), a sequence that will stabilize the viral polypeptide (e.g., albumin), a sequence that will aid in the purification of the viral polypeptide (e.g., streptavidin, biotin, and poly-His tags), or a sequence that will target the viral polypeptide to autoreactive immune cells (autoreactive CD8⁺ T cells) (e.g., an anti-CD8 antibody or antibody fragment).

The non-virus-derived polypeptide(s) may be covalently joined to the N- and/or C-terminus of the viral polypeptide or may be non-covalently associated with the viral polypeptide. For example, the non-virus derived polypeptide may associate with the viral polypeptide through ionic bonds or hydrophobic interactions. The non-virus derived polypeptides may also be chemically-crosslinked to the viral polypeptide(s) in the composition.

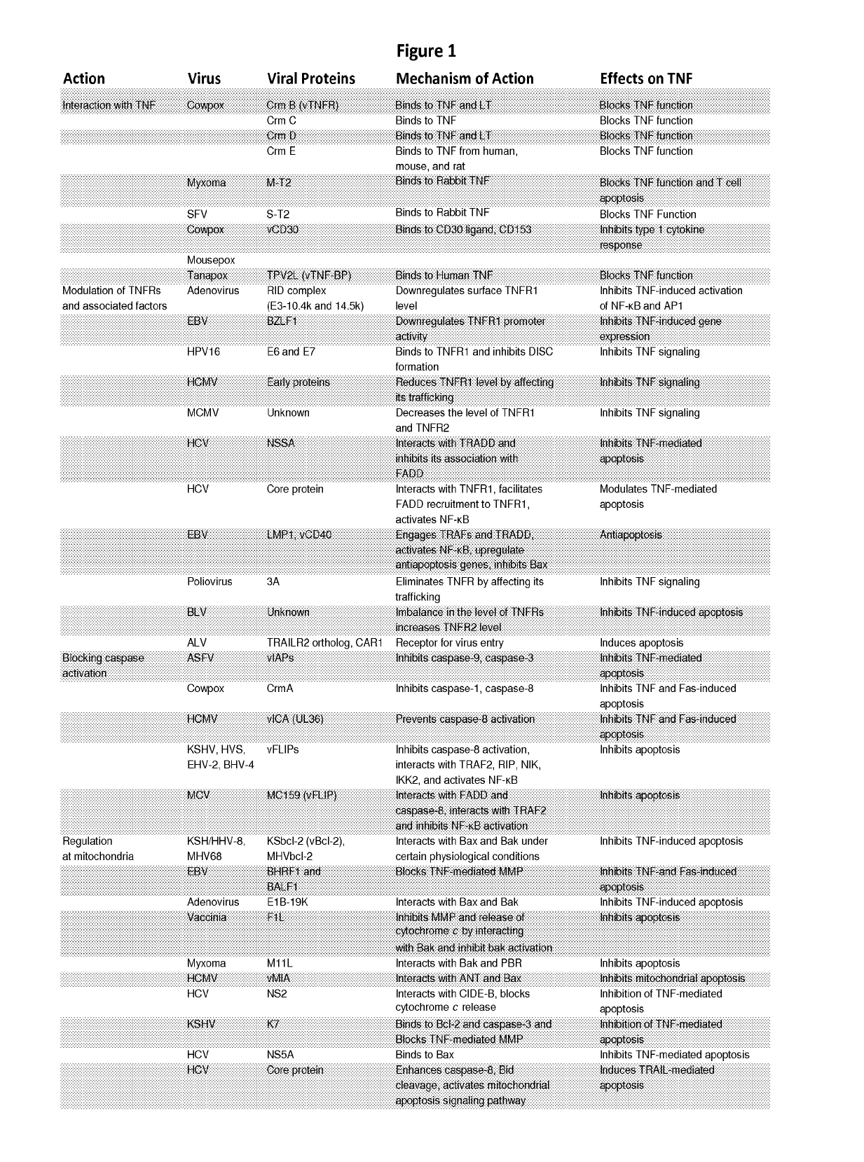

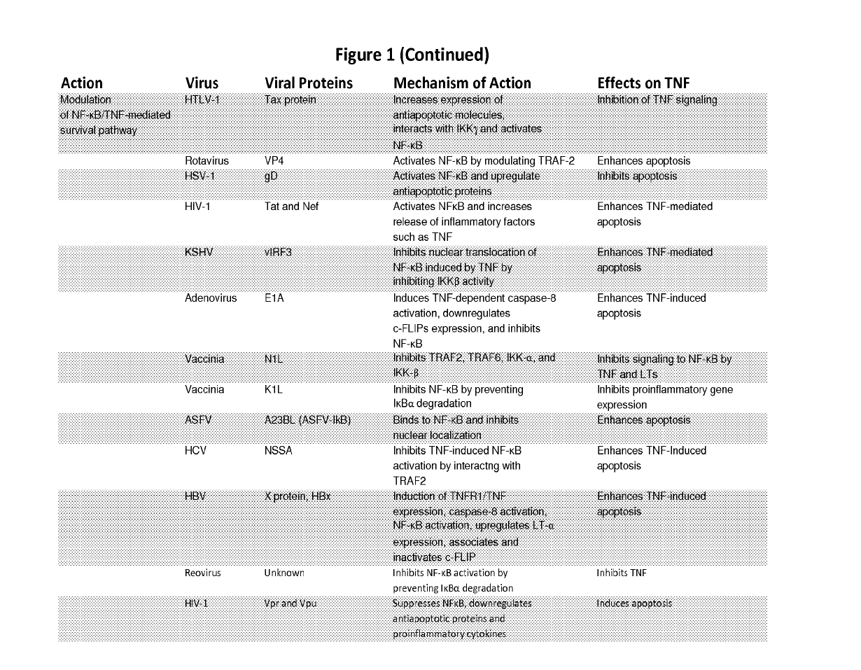

The compositions of the invention may contain all or a portion of one or more live, killed, attenuated, or inactivated virus(es). Desirably, the compositions of the invention contain a live, killed, attenuated, or inactivated virus (e.g., a DNA or RNA virus) that stimulates TNF-α expression, activates TNF-α signaling pathways, activates or results in the activation of the TNF-α receptor (e.g., TNF-α receptor 2), and/or activates NF-κB pathways in a mammalian cell (e.g., a human cell). Non-limiting examples of DNA and RNA viruses that may be included in the compositions of the invention are listed in Tables 1 and 2. Additional examples of viruses that may be included in the compositions of the invention are listed in FIG. 1 (from Rahman et al., Plos Pathogens 2:e4, 2006); these viruses may be modified, if necessary, to ablate or modify viral polypeptides that prevent TNF-α receptor binding or otherwise prevent signaling through the TNF-α receptor, as discussed below.

DNA or RNA viruses included in the compositions of the invention (e.g., the viruses listed in Table 1, Table 2, and FIG. 1), whether live, killed, attenuated, or inactivated, may be modified to contain a mutation that decreases an activity that will otherwise block or inhibit the stimulation of TNF-α expression, activation of TNF-α signaling pathways, activation of TNF-α receptor(s), and/or activation of NF-κB pathways in a mammalian cell (in vitro or in vivo). For example, several viruses express a soluble protein that mimics the TNF-α receptor, and thus decreases the activity of TNF-α or the activation of TNF-α signaling pathways in a cell (see, e.g., Rahman et al., Plos Pathogens 2:e4, 2006). The use of DNA or RNA viruses that are mutated to ablate these types of proteins or functions are envisioned in the compositions and methods of the invention.

Several live, killed, attenuated, or inactivated DNA and RNA viruses (e.g., the one or more DNA and RNA viruses listed in Table 1, Table 2, and FIG. 1) are commercially available. Methods for the preparation and purification of live, killed, attenuated, or inactivated viruses are also known in the art (see, e.g., U.S. Pat. Nos. 6,511,667; 7,052,701; and 7,494,659; each of which is herein incorporated by reference). Tables 1 and 2 also describe strategies for treatment of autoimmune diseases in a mammal (e.g., a human) using the listed DNA and RNA viruses, whether live, killed, attenuated, or inactivated.

TABLE 1
DNA Viruses

| | | | | Treatment Strategy |
|---|---|---|---|---|
| | | | Viral Defense to Inhibit | (Live or Attenuated) |
| | | Protective | Protective TNF or Viral | Virus or Portions of |
| | | Normal | Response to Produce | a Virus with These |
| DNA | Virus Name | Host | Proliferation | Additional Traits |
| Acute | Poxviridae | ↑ TNF | Viral TNF receptors (mimics of | Poxviridae infection |
| | | | host TNFR, e.g., T2 protein) to | with deletion or |
| | | | prevent host TNF | inactivation of T2 |
| | | | | proteins or |
| | | | | inactivation of viral |
| | | | | TNF receptors |
| Acute | Vaccinia | ↑ TNF | BBR gene prevents TNF | Vaccinia without BBR |
| | | | mediated apoptosis | gene |
| Acute | Tanapox Virus | ↑ TNF | 38 kDa-glycoprotein inhibits | Tanapox Virus |
| | | | TNF induced activation of | without 38 kDa protein |
| | | | NFκB | |
| Acute or | Herpes SV | ↑ TNF | Virus blocks caspase-3- | HSV with inactivation |
| Chronic | | | mediated cell death, therefore | of caspase-3 blockage. |
| | | | preventing TNF from killing | HSV infected cells |
| | | | infected cells | make HVEM that |
| | | | | helps virus entry with |
| | | | | virus made GD |
| | | | | protein. HVEM is a |
| | | | | mimic of TNFR and |
| | | | | therefore as a |
| | | | | therapeutic should be |
| | | | | deleted. HVEM and |
| | | | | GD activate the |
| | | | | TRAF1, 2 pathway |
| | | | | and therefore would |
| | | | | be benefical |
| | | | | therapeutics. |
| Chronic/ | EBV | ↑ TNF | Virus tries to produce chronic | Drugs made from |
| Cancer | | | infection by LMP1. LMP1 is a | EBV, EBV-LMP1, |
| Causing | | | pseudo-TNFR that activates | LMP1 C-terminal |
| | | | NFκB through C-terminal | domain only. LMP1 |
| | | | domain. Virus with LMP1 | acts like a TNFR2 |
| | | | stimulates NFκB for | stimulator and would |
| | | | lymphoproliferative or | be a therapeutic target |
| | | | hemaphagocytic syndromes. | |
| Chronic | CMV | ↑ TNF | For latency CMV tries to | TNF stimulates IE |
| | | | stimulate TNF by IE genes | enhancer/promoter |
| | | | | activity in immature |
| | | | | cells only where they |
| | | | | have trans-activation |
| | | | | function, such as TNF |
| | | | | genes. [Therapy: |
| | | | | Treatment with IE |
| | | | | enhancer/promoter |
| | | | | genes from CMV]. |
| Chronic/ | Herpesvirus | ↑ TNF | This virus tries for a cancer | Treatment with STP |
| Cancer | Saimiri (HVS) | | strategy, i.e., activation of | proteins. Treatment |
| Causing | | | NFκB by STP oncoproteins | with inactive virus. |
| | | | | Residues of STP |
| | | | | proteins such as |
| | | | | PXQXT/S in STP- |
| | | | | ALL association with |
| | | | | TRAF. STP C 488 |
| | | | | treatment would |
| | | | | activate NFκB and be |
| | | | | a therapeutic |
| Chronic | Hepatitis B Virus | ↑ TNF | This virus tries for chronic | HBx protein sensitizes |
| | (HBV) | | infections | cells to apoptotic |
| | | | | killing by TNF, thus |
| | | | | treatment with HBx |
| Chronic/ | Parvovirus H1 | ↑ TNF | Virus tries to activate caspase-3, | Infect with a virulent |
| Cancer | | | i.e., apoptosis to release more | version. Activation of |
| Causing | | | viral particles - may do this by | c-Myc |
| | | | inhibiting c-Myc | |

TABLE 2
RNA Viruses

| | | | | |
|---|---|---|---|---|
| RNA Viruses | HIV | ↑ TNF | HIV virus makes HIV tat | Treatment with |
| | | | which activates TNF | inactivated HIV or |
| | | | signaling through NFκB | HIV Tat to |
| | | | | stimulate NFκB |
| | | | | signaling |
| | HCV | ↑ TNF | HCV core protein inhibits | Immunize with |
| | (Hepatitis) | | TNF apoptosis | HCV without core |
| | | | | protein, produce |
| | | | | inhibitors of HCV |
| | | | | core protein. |
| | | | | Immunize with |
| | | | | HCV without NS5A |
| | | | HCV core proteins bind to | Make core |
| | | | TNFR1 and enhance | analogues that bind |
| | | | apoptosis | to 345-407 of the |
| | | | | death domain of |
| | | | | TNFR1 |
| | Influenza | ↑ TNF | TNF directly produced by | Influenza probably |
| | | | influenza infected cells. | stimulates TNF by |
| | | | | LPS and GM-CSF |
| | | | TNF also triggered by | Immunize with the |
| | | | influenza viral | inactive virus. Also |
| | | | nucleopeptide SDYEGRLI | virion surface |
| | | | with bound class I | hemagglutinin can |
| | | | | directly activate |
| | | | | NFκB. |
| | RSV | ↑ TNF | Virus promotes persistent | Viral infections |
| | | | NFκB. Virus may also | especially with |
| | | | prevent apoptosis by the | viruses that have |
| | | | cysteine noose of RSV-G. | inhibition or |
| | | | | deletion/mutation of |
| | | | | RSV cysteines. |
| | Measles | ↑ TNF | | Inactive virus |
| | Vesicular | ↑ TNF | | Inactive virus |
| | stomalitis virus | | | |
| | (VSV) | | | |
| | Dengue | | | Inactive virus |
| | Ebola | | | Inactive virus |

Compositions of the invention that contain a viral polypeptide or nucleic acid encoding a viral polypeptide, as described above, may also contain TNF-α or one or more (e.g., 1, 2, 3, 4, or 5 or more) TNF-α receptor agonists, one or more intracellular mediators of the TNF-α signaling pathway (e.g., 1, 2, 3, 4, or 5 or more), or one or more TNF-α inducing substances. A TNF-α receptor agonist is a molecule (e.g., a small molecule, a polypeptide, and an antibody) that binds and/or activates a TNF-α receptor (e.g., TNF-α receptor 1 or TNF-α receptor 2). Non-limiting examples of TNF-α receptor agonists include small molecules (e.g., the small molecule agonists described in Hymowitz et al., Nature Chem. Biol. 1:353-354, 2005) and antibodies that bind to the TNF-α receptor (e.g., clones MR2-1 (TNFR2), MR1-2 (TNFR1), and 80M2 (TNFR2) (Cell Sciences HM2022) from Cell Sciences, and Sigma T1815 (clone 22221.311)(TNFR2)). Non-limiting examples of one or more intracellular mediators of the TNF-α signaling pathway are described in Karin et al. (Nature Reviews Drug Discovery 3:17-26, 2004) and include TRAFF, TRAD, RIP, Rel (RelA), NFκB, IKKα, IKKγ, FADD, and MEKK1/7. Non-limiting examples of TNF-α inducing substances include complete Freund's adjuvant, BCG, tissue plasminogen factor, LPS, interleukin-1, interleukin-2, lymphotoxin, and cachectin.

Methods for the expression and purification of recombinant proteins, such as the viral polypeptides having at least 80% or more sequence identity to a naturally-occurring viral polypeptide or a fragment or fusion protein thereof are known in the art. For example, expression of the recombinant protein may be performed in competent bacterial or yeast strains, or expression may be performed in transgenic mammals (e.g., cows or goats) with the recombinant protein expressed in, e.g., the serum or milk of the mammal. Purification of the recombinant proteins may be performed using standard techniques known in the art, including, but not limited to precipitation, size exclusion, and/or column chromatography methods, and may also include a step of affinity chromatography when the recombinant protein has been designed to contain an affinity moiety (e.g., a His₆ tag or streptavidin tag). Desirably, the one or more (e.g., 1, 2, 3, 4, or 5 or more) viral polypeptides or the nucleic acid encoding one or more (e.g., 1, 2, 3, 4, or 5 or more) viral polypeptides is/are at least 80%, 85%, 90%, 95%, 96%, 97%, 98%, 99%, or even 100% pure from other cellular components (e.g., other cellular proteins or nucleic acids).

The nucleic acid(s) encoding a viral polypeptide of the invention may be cloned into a vector that, optionally, is operably linked to a control sequence which is capable of providing for the expression of the encoded viral polypeptide by the host cell, e.g., an expression vector. The term “operably linked” refers to a juxtaposition wherein the components described are in a relationship permitting them to function in their intended manner. A regulatory sequence, such as a promoter, “operably linked” to a coding sequence is positioned is such a way that the expression of the coding sequence is achieved under conditions compatible with the regulatory sequence.

The nucleic acid(s) encoding a viral polypeptide may be administered by means of specialized delivery vectors using gene therapy. Gene therapy methods are discussed in Verme et al. (Nature 389:239-242, 1997). Both viral and non-viral vector systems can be used. The vectors may be, for example, plasmids, artificial chromosomes (e.g., bacterial, mammalian, or yeast artificial chromosomes), virus or phage vectors provided with a origin of replication, and optionally, a promoter for the expression of the nucleic acid encoding the viral polypeptide and optionally, a regulator of the promoter. The vectors may contain one or more selectable marker genes, for example, an ampicillin or kanamycin resistance gene in the case of a bacterial plasmid or a resistance gene for a fungal vector. Vectors may be used in in vitro, for example, for the production of DNA, RNA, or the viral polypeptide, or may be used to transfect or transform a host cell, for example, a mammalian host cell, e.g., for the production of the viral polypeptide encoded by the vector. The vectors may also be adapted to be used in vivo, for example, in a method of vaccination or gene therapy.

Examples of suitable viral vectors include, retroviral, lentiviral, adenoviral, adeno-associated viral, herpes viral, including herpes simplex viral, alpha-viral, pox viral, such as Canarypox and vaccinia-viral based systems. Gene transfer techniques using these viruses are known in the art. Retrovirus vectors, for example, may be used to stably integrate the nucleic acids of the invention into the host genome. Replication-defective adenovirus vectors by contrast remain episomal and therefore allow transient expression. Vectors capable of driving expression in insect cells (e.g., baculovirus vectors), in human cells, yeast, or in bacteria may be employed in order to produce quantities of the viral polypeptide(s) encoded by the nucleic acids of the invention, for example, for use in subunit vaccines or in immunoassays.

In one example, a replication-deficient simian adenovirus vector may be used as a live vector. These viruses contain an E1 deletion and can be grown on cell lines that are transformed with an E1 gene. Examples of these replication-deficient simian adenovirus vectors are described in U.S. Pat. No. 6,083,716 and WO 03/046124 (each of which is herein incorporated by reference). These vectors can be manipulated to insert a nucleic acid of the invention, such that the encoded viral polypeptide(s) may be expressed.

Promoters and other expression regulatory signals may be selected to be compatible with the host cell for which expression is designed. For example, mammalian promoters include the metallothionein promoter, which can be induced in response to heavy metals such as cadmium, and the β-actin promoter. Viral promoters, such as the SV40 large T antigen promoter, human cytomegalovirus (CMV) immediate early (1E) promoter, rous sarcoma virus LTR promoter, adenovirus promoter, or a HPV promoter, particularly the HPV upstream regulatory region (URR) may also be used. All these promoters, as well as additional promoters, are well-described in the art.

The nucleic acid(s) of the invention may also be administered using non-viral based systems. For example, these administration systems include microsphere encapsulation, poly(lactide-co-glycolide), nanoparticle, and liposome-based systems.

The above viral polypeptides and nucleic acids of the invention may be used to treat an autoimmune disease in a mammal (e.g., a human). Examples of autoimmune diseases that may be treated by the methods of the invention, without limitation, include alopecia areata, ankylosing spondylitis, antiphospholipid syndrome, autoimmune Addison's disease, autoimmune hemolytic anemia, autoimmune hepatitis, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatricial pemphigoid, CREST syndrome, cold agglutinin disease, Crohn's disease, discoid lupus, systemic lupus erythmatosous, ulcerative colitis, psoriatic arthritis, essential mixed cryoglobulinemia, fibromyalgia-fibromyositis, Graves' disease, Guillain-Barré, Hashimoto's thyroiditis, hypothyroidism, idiopathic pulmonary fibrosis, idiopathic thrombocytopenia purpura (ITP), IgA nephropathy, insulin-dependent diabetes, juvenile arthritis, lichen planus, lupus, Ménière's disease, mixed connective tissue disease, multiple sclerosis, myasthenia gravis, pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjögren's syndrome, Stiff-Man syndrome, Devic's disease, Takayasu arteritis, temporal arteritis/giant cell arteritis, ulcerative colitis, uveitis, vasculitis, vitiligo, and Wegener's granulomatosis. The methods of the invention provide for the treatment of patients with new-onset or recently diagnosed autoimmune disease (e.g., those patients diagnosed with, or that develop one or more symptoms of, an autoimmune disease within 2 weeks, 1 month, two months, three months, four months, six months, one year, or two years) or patients with long-term or established autoimmune disease (e.g., a mammal diagnosed with or having one or more symptoms of an autoimmune disease over two years, five years, ten years, fifteen years, twenty years, twenty-five years, thirty years, thirty-five years, or forty years).

### Animal Models and Biological In Vitro Assays for Testing the Compositions of the Invention

The efficacy or activity of the compositions containing a viral polypeptide or a nucleic acid encoding a viral polypeptide may be tested using in vitro, in vivo, and animal model assays. In vitro assays may be performed to determine whether the compositions of the invention containing a viral polypeptide or a nucleic acid encoding a viral polypeptide (e.g., a purified viral polypeptide or live or attenuated DNA or RNA virus that stimulates TNF-α expression) mediate an increase in TNF-α expression (e.g., TNF-α protein or TNF-α mRNA), activate TNF-α signaling pathways, agonize the TNF-α receptor, or activate NF-κB signaling pathways in a mammalian cell. The compositions of the invention may also be tested in vitro for their ability to induce autoreactive CD8⁺ T cell death (see, e.g., the methods described in Ban et al., Proc. Natl. Acad. Sci. 105:13644-13649, 2008). Also, the compositions of the invention can be tested in vitro for the ability to augment (e.g., increase) cultured human CD4⁺ T cells Treg function, numbers, or phenotype.

The compositions of the invention may also be tested in vivo for their ability to induce autoreactive immune cell (autoreactive CD8⁺ T cell) death (e.g., apoptosis), to increase the number of T regulatory cells, to reduce the production of autoantibodies, and to increase the number (and/or biological activity) of the cells or tissue in a mammal targeted by the autoimmune disease or a biological activity mediated by the cells or tissue. For example, there are several animal models of autoimmune diseases available in the art, e.g., insulin-dependent diabetes (non-obese diabetic mice (NOD mice)), BB rats (BioBreeding Laboratories), lupus ((NZB×NZW)F1 or MRL/lpr mice), and rheumatoid arthritis (collagen-induced arthritis in DBA/1 mice). These animal models may be used to determine the efficacy of the compositions of the invention to treat an autoimmune disease. As described above, the treated animals may be observed for a reduction in the severity or the alleviation of one or more symptoms of an autoimmune disease. In addition, the level of induced autoreactive immune cell death (autoreactive CD8⁺ T cell apoptosis), the level of T regulatory cells, and the levels of autoantibodies, as well as the number and biological activity of the targeted cells and tissues may be assessed in the treated animals, and compared to the values observed in control animals (e.g., control animals that do not receive treatment or a biological sample obtained from an animal prior to treatment).

### Pharmaceutical Compositions of the Invention

The compositions of the invention (e.g., compositions containing a viral polypeptide or a nucleic acid molecule encoding a viral polypeptide or compositions that include an immunosuppressive agent (e.g., an anti-CD antibody, such as teplizumab and/or otelixizumab) may be formulated using any of the methods known in the art for parenteral, intravenous, intra-arterial, subcutaneous, intramuscular, intraorbital, intraventricular, topical, intraspinal, intraperitoneal, intradermal, intranasal, intracranial, or oral administration. The compositions of the invention may include one or more (e.g., 1, 2, 3, 4, or 5 or more) viral polypeptides or a nucleic acid encoding one or more (e.g., 1, 2, 3, 4, or 5 or more) viral polypeptides or one or more immunosuppressive agents (e.g., 1, 2, 3, 4, or 5 or more). The compositions may further contain one or more (e.g., 1, 2, 3, 4, or 5 or more) non-virus-derived polypeptides, one or more (e.g., 1, 2, 3, 4, or 5 or more) TNF-α receptor agonists, one or more (e.g., 1, 2, 3, 4, or 5 or more) intracellular mediators of the TNF-α signaling pathway, and/or one or more (e.g., 1, 2, 3, 4, or 5 or more) TNF-α inducing agents.

The compositions may be administered to a mammal (e.g., a human) prior to the development of symptoms of the autoimmune disease (i.e., an asymptomatic mammal) or the compositions may be administered to the patient after diagnosis with an autoimmune disease or after presentation with one or more (e.g., 1, 2, 3, 4, or 5) symptoms of an autoimmune disease (i.e., in a patient with short-term disease (new-onset or recent diagnosis of autoimmune disease), e.g., diagnosis or development of one or more symptoms of an autoimmune disease within 2 weeks, 1 month, two months, three months, four months, six months, one year, or two years; or long-term or established autoimmune disease, e.g., a mammal diagnosed with or having one or more symptoms of an autoimmune disease over two years, five years, ten years, fifteen years, twenty years, twenty-five years, thirty years, thirty-five years, or forty years). In addition, a composition that includes an immunosuppressive agent, such as an anti-CD antibody (e.g., teplizumab and/or otelixizumab) may be administered to treat an autoimmune disease in a mammal in which an EBV infection has been detected.

The compositions may be administered to a mammal (e.g., a human) in one or more doses (e.g., 2, 3, 4, 5, 6, 7, 8, 9, or 10 or more doses). If more than one dose is administered, the doses may be administered via the same mode of administration (e.g., intravenous or intradermal administration) or by different modes of administration (e.g., intravenous and intramuscular administration). The mammal may also be administered different doses at different times. For example, the mammal may be administered a higher initial dose and lower subsequent doses over the course of treatment.

A dose of the composition may be administered daily, twice daily, three times daily, weekly, bi-weekly, every three weeks, monthly, bimonthly, every three months, every four months, every six months, once per year, or once every two years. The dose of the composition may be determined by a skilled physician upon consideration of a subject's clinical symptoms and/or physical condition (e.g., weight, sex, height, and severity of the autoimmune disease). The composition may be administered by parenteral, intradermal, intravenous, intra-arterial, subcutaneous, intramuscular, intraorbital, topical, intraventricular, intraspinal, intraperitoneal, intranasal, intracranial, or oral administration. The composition (e.g., a composition containing a viral polypeptide or a nucleic acid molecule encoding a viral polypeptide or a composition that includes an immunosuppressive agent (e.g., an anti-CD antibody, such as teplizumab and/or otelixizumab)) may be administered, for example, at a dose of, e.g., 10 ng to 1 μg, 100 ng to 1 μg, 1 μg to 10 μg, 5 μg to 100 μg, 50 μg to 200 μg, 100 μg to 500 μg, 2 μg to 30 mg, 30 μg to 1 mg, 300 μg to 1 mg, 0.5 mg to 2 mg, 1 mg to 10 mg, 5 mg to 50 mg, 25 mg to 100 mg, 50 mg to 250 mg, 100 mg to 500 mg, or 400 mg to 1 g.

For compositions that contain a live, killed, attenuated, or inactivated virus, the composition may be administered to a mammal in one or more doses (e.g., 1, 2, 3, 4, or 5 or more doses) at weekly, bi-weekly, tri-weekly, monthly, bi-monthly, tri-monthly, half-year, yearly, or bi-yearly intervals. The virus may be administered using parenteral, intradermal, intravenous, intra-arterial, subcutaneous, intramuscular, intraorbital, topical, intraventricular, intraspinal, intraperitoneal, intranasal, intracranial, or oral administration. A skilled physician may determine the most appropriate route of administration and dose of the virus to be administered upon consideration of a subject's clinical symptoms and/or physical condition (e.g., weight, sex, height, and severity of the autoimmune disease). A composition containing a live or attenuated virus may be administered at a dose of 1×10³ to 1×10¹¹ pfu, 1×10³ to 1×10⁴ pfu, 5×10³ to 5×10⁴ pfu, 1×10⁴ to 1×10⁶ pfu, 1×10⁵ to 1×10⁷ pfu, 1×10⁶ to 1×10⁸ pfu, 1×10⁷ to 1×10⁹ pfu, or 1×10⁸ to 1×10¹¹ pfu. Commercially available viruses may also be administered according to the manufacturer's instructions.

The compositions of the invention may be prepared in a pharmaceutically acceptable carrier or excipient. Such suitable carriers or excipients are, for example, water, phosphate-buffered saline (PBS), acetate-buffered saline (ABS), Ringer's solution, dextrose, glycerol, ethanol, or the like and combinations thereof. In addition, if desired, a composition for administration to a mammal can contain minor amounts of auxiliary substances such as wetting or emulsifying agents, or pH buffering agents that enhance the effectiveness of the composition. The compositions of the invention may also be prepared in any acceptable salt formulation.

### Combination Therapies

The methods of treatment described herein may also include the administration of one or more additional agents for the treatment of an autoimmune disease that are known in the art. For example, the administration of a composition containing a viral polypeptide or a nucleic acid molecule encoding one or more viral polypeptides or a composition containing an immunosuppressive agent (e.g., an anti-CD antibody, such as teplizumab and/or otelixizumab), as described herein, may be administered to a mammal in conjunction with one or more additional substances that induce TNF-α expression or activity, for example, complete Freund's Adjuvant, ISS-ODN, microbial adjuvants, such as cell wall components with LPS-like activity, cholera particles, E. coli heat labile enterotoxin, E. coli heat labile enterotoxin complexed with lecithin vesicles, ISCOMS-immune stimulating complexes, chemical adjuvants, such as polyethylene glycol and poly(N-2-(hydroxypropyl)methacrylamide), synthetic oligonucleotides containing CpG or CpA motifs, lipid A derivatives, such as monophosphoryl lipid A, monophosphoryl lipid (MPL), muramyl dipeptide derivatives, BCG, tissue plasminogen activator (TPA), lipopolysaccharide (LPS), interleukin-1, interleukin-2, UV light, lymphotoxin, cachectin, a TNFR-2 agonist (e.g., a TNFR-2 agonist antibody), a NF-κB inducing substance, lymphotoxin, IRF-1, STAT1, an agonist of an ICS-2IgAS promoter element, or the combination of TNF-α and a TNFR-1 antibody. Preferably, the additional substance is BCG.

The provided methods may also include administration of one or more agents that activate an intracellular mediator of a TNF-α signaling pathway. For example, the methods of the invention may also include administration of one or more polypeptides, small molecules, or antibodies that bind to or activate NF-κB, Jun N-terminal kinase, TRAILR2, FasL, TRADD, FADD, TRAF2, RIP, MAPK, kinase activators, a caspase, or a pro-caspase. The methods of the invention may also include one or more polypeptides, small molecules, or antibodies that bind to or activate one or more members of the TNF receptor superfamily, such as, TNF receptor 1 or 2, Trail-R1, Trail-R2, Trail-R3, Trail-R4, OPG, Rank, Fn14, DR6, Hvem, LtbetaR, DcR3, Tramp, Fas, CD40, CD30, CD27, 4-1BB, OX40, Gitr, Ngfr, BCMA, Taxi, Baff-4, EDAR, Xedar, Troy, Relt, or CD95L. In addition, the methods of the invention may further include administering one or more polypeptides or antibodies that function as TNF super family ligands, for example, TRAIL-R1 (DR4), TRAIL-R2 (DR5), TRAIL-R3 (DCR1), TRAIL-R4 (DCR2), OPG, RANK, RN14, DR6, THF-R2 (CD120B), TNF-R1 (CD120A), FVEM, LIBETAR, CDR3, TRAMP (DR3), FAS(CD95), CD40, CD30, CD27, 4-1BB(CD137), CD134(OX40), GITR, NGFR, RIP, BCMA, TACI, BAFFR, EDAR, XEDAR, TROY, or RELT. The methods of the invention may also include administration of one or more of the following to a mammal: IL-1, IL-2, IL-4, IL-5, IL-6, IL-7, IL-10, IL-11, IL-12, IL-13, IL-18, IFN-α, IFN-γ, TGF-β, PDGF, or VEGF. The methods of the invention may also include the administration of a small molecule or antibody agonist of one or more of TLR1, TLR2, TLR6, TLR3, TLR4, TLR5, TLR7, and/or TLR9.

The additional polypeptides, small molecules, or antibodies described above may be administered at different times than the compositions containing the viral polypeptide(s) or the nucleic acid(s) of the invention or the compositions containing an immunosuppressive agent (e.g., an anti-CD antibody, such as teplizumab and/or otelixizumab), or at the same time, or they may be administered in the same or different dosage forms.

### Diagnosis of Autoimmune Disease

A mammal may be diagnosed with an autoimmune disease by a physician using methods known in the art (e.g., molecular diagnostic tests and/or examination for the clinical symptoms of an autoimmune disease; see, e.g., U.S. Patent Publication No. 2004/0229785). The clinical symptoms of an autoimmune disease depend on the specific tissue targeted by the autoreactive immune cells in the mammal (e.g., β-islet cells in type 1 diabetes, thymocytes in Hashimoto's disease, and mucosal epithelial cells in Crohn's disease). Non-limiting examples of symptoms of autoimmune diseases include increased levels of autoantibodies, increased levels of autoreactive immune cells (autoreactive CD8⁺ T cells), loss of targeted cells or targeted tissue damage, fatigue, depression, sensitivity to cold, weight gain, muscle weakness, constipation, insomnia, irritability, weight loss, weight gain, bulging eyes, muscle tremors, skin rashes, painful or swollen joints, sensitivity to the sun, loss of coordination, and paralysis.

Specific examples of symptoms of insulin-dependent diabetes include decreased pancreatic β-cell number, decreased insulin production, decreased C-peptide levels, increased glycated hemoglobin levels (i.e., hemoglobin A1c levels or HbA1c levels), increased thirst, frequent urination, extreme hunger, weight loss, fatigue, blurred vision, diabetic ketoacidosis (DKA), increased insulin-specific autoreactive immune cells (autoreactive CD8⁺ T cells), decreased T regulatory cells, and increased levels of autoantibodies (e.g., increased levels of anti-glutamic acid dehydrogenase and anti-pancreatic beta cell-specific zinc transporter antibodies).

A mammal to be treated using the methods of the invention may be identified as being at risk for the development of an autoimmune disease (e.g., having at least a 5%, 10%, 15%, 20%, 25%, 30%, 40%, or 50% increased chance of developing an autoimmune disease) using molecular genetic methods known in the art to identify genetic defects in the mammal's CD8⁺ T cells or by analysis of the medical history of the mammal's family.

### Methods of Assessing Treatment of Autoimmune Diseases

Several methods can be used to assess the efficacy of treatment according to the methods of the invention. For example, treatment of an autoimmune disorder according to the methods of the invention may result in at least a 1% decrease (e.g., at least a 10%, 15%, 20%, 25%, 30%, 35%, 40%, 45%, 50%, 55%, 60%, 70%, 80%, 90%, or even 100% decrease) in the severity of, or alleviation in, one of more (e.g., 1, 2, 3, 4, or 5) symptoms of an autoimmune disease. Treatment may result in the reverenation of targeted cells or tissue damaged by the autoimmune disease, or in a reduction in fatigue, depression, sensitivity to cold, weight gain, muscle weakness, constipation, insomnia, irritability, weight loss, weight gain, bulging eyes, muscle tremors, skin rashes, painful or swollen joints, sensitivity to the sun, loss of coordination, and paralysis. The efficacy of treatment of an autoimmune disease according to the present methods may also be shown by observation of at least a 1% increase (e.g., at least a 2%, 3%, 4%, 5%, 6%, 7%, 8%, 9%, 10%, 15%, 20%, 25%, 30%, 35%, 40%, or 50% increase) in autoreactive CD8⁺ T cell death (e.g., apoptosis) in a mammal compared to a control mammal or a control sample (e.g., a mammal with an autoimmune disease not receiving the treatment or a biological sample from a mammal prior to the start of treatment). Treatment of an autoimmune disease according to the present methods may also result in at least a 10% decrease (e.g., at least a 15%, 20%, 25%, 30%, 35%, 40%, 45%, 50%, 55%, 60%, 65%, 70%, 75%, 80%, 85%, 90%, or even 100% decrease) in the autoantibody levels in a mammal relative to the autoantibody levels in control mammal or control sample (e.g., a mammal with an autoimmune disease not receiving the treatment or a biological sample from a mammal prior to the start of treatment). Treatment of an autoimmune disease may also result in at least a 0.1% increase (e.g., at least a 0.2%, 0.3%, 0.4%, 0.5%, 0.6%, 0.7%, 0.8%, 0.9%, 1%, 2%, 3%, 5%, 10%, 20%, 30%, 40%, or 50% increase) in the number of regulatory T cells in a mammal compared to a control mammal or control sample (e.g., a mammal with an autoimmune disease not receiving the treatment or a biological sample from a mammal prior to the start of treatment). In addition, the successful treatment of an autoimmune disease may provide for the regeneration of the targeted cells or tissues, or an increase (e.g., at least a 1%, 5%, 10%, 15%, 20%, 25%, 30%, 35%, 40%, 45%, 50%, 55%, 60%, 70%, 80%, 90%, or even 100% increase) in one or more (e.g., 1, 2, 3, 4, or 5) biological activities of the targeted cells (e.g., insulin production by islet cells in a type 1 diabetic following treatment according to the present methods) or tissue within a mammal.

A number of different methods for assessing treatment efficacy in insulin-dependent diabetics are also known in the art. For example, successful treatment of insulin-dependent diabetes will result in at least a 1% decrease (e.g., at least a 10%, 15%, 20%, 25%, 30%, 35%, 40%, 50%, 60%, 70%, 80%, 90%, or even 100% decrease) in one or more (e.g., 1, 2, 3, 4, or 5) symptoms of insulin-dependent diabetes, such as thirst, frequent urination, extreme hunger, weight loss, fatigue, blurred vision, and ketoacidosis (DKA). In addition, treatment of insulin-dependent diabetes desirably results in at least a 1% increase in pancreatic β-cell number (e.g., at least 5%, 10%, 15%, 20%, 25%, 30%, 35%, 40%, 45%, 50%, 55%, 60%, 70%, 80%, 90%, or even 100% increase in the pancreatic (3-cell number relative to a control mammal not receiving the treatment), at least a 5% increase (e.g., at least 10%, 15%, 20%, 25%, 30%, 35%, 40%, 45%, 50%, 55%, 60%, 70%, 80%, 90%, or even 100% increase) in insulin production, at least a 1% increase (e.g., at least 5%, 10%, 15%, 20%, 25%, 30%, 35%, 40%, 45%, 50%, 55%, 60%, 70%, 80%, 90%, or even 100% increase) in C-peptide levels, at least a 1% decrease in glycated hemoglobin A1c levels (e.g., at least 10%, 15%, 20%, 25%, 30%, 35%, 40%, 45%, 50%, 60%, 70%, 80%, 90%, or even 100%), at least a 1% increase (e.g., 2%, 3%, 4%, 5%, 6%, 7%, 8%, 9%, 10%, 15%, 20%, 25%, 30%, 35%, 40%, or 50% increase) in autoreactive CD8⁺ T cell (e.g., autoreactive insulin CD8⁺ T cell) death (e.g., apoptosis), or at least a 0.1% increase in the number of regulatory T cells in the mammal (at least a 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 10%, 20%, 30%, 40%, or 50% increase) relative to a control mammal or control sample (e.g., a mammal having insulin-dependent diabetes that does not receive treatment or a sample from the mammal having insulin-dependent diabetes prior to treatment). Treatment of a mammal having insulin-dependent diabetes also desirably results in at least a 5% decrease (e.g., at least a 10%, 15%, 20%, 25%, 30%, 35%, 40%, 45%, 50%, 55%, 60%, 65%, 70%, 75%, 80%, 85%, 90%, or even 100% decrease) in the levels of anti-glutamic acid dehydrogenase (anti-GAD) auto-antibodies or the levels of anti-pancreatic beta cell-specific zinc transporter (anti-ZnT8A) auto-antibodies.

### Serological Testing for EBV Infection

Serological testing can be used to detect EBV infection (e.g., an acute, chronic, subclinical, or latent infection, or an infection due to reactivation) in a mammal (e.g., a human). For example, EBV infection may be determined using an antibody-based assay or a PCR-based assay.

Infectious mononucleosis (IM), which is caused by EBV infection, is characterized by a triad of symptoms: fever, pharyngitis, and adenopathy. Diagnosis of IM can be made using, e.g., a hematological, serological, and/or symptomatic assessment of the patient. For example, diagnosis of IM can be made by demonstrating the presence of IgM antibodies directed against the causative agent, the Epstein-Barr virus.

Hematological indicators of IM also include the rise in white blood cell count to approximately 10-15,000 cells per mm² in the first 2 to 3 weeks of the disease. This leads to lymphocytosis in approximately 70% of cases. Both B and T lymphocytes contribute to 10-30% of the characteristic increase in atypical lymphocytes but in older patients this increase is not as marked. Compared with normal lymphocytes, these atypical cells are generally larger with a distorted shaped nucleus. The lymphocytes are not exclusive to IM and may be associated with other disease states such as that caused by cytomegalovirus, viral hepatitis, measles, rubella, and drug reactions. In addition to lymphocytosis, more than 50% of patients develop mild thromobocytopenia.

Infection with EBV is characterised by the development of specific antibodies to antigenic components of the virus. These antigens appear at different stages of infection and differ in lytic versus latent infection. Antibodies to EBV antigens measured for clinical purposes are those to viral capsid antigen (VCA), early antigens (EA), and Epstein-Barr nuclear antigen (EBNA). EA are expressed early in the lytic cycle, while VCA and membrane antigens are structural proteins expressed late in the lytic cycle. EBNA is expressed in cells that are latently infected. Antibodies to these proteins are measured by enzyme immunoassays, indirect immunofluorescence assays, and immunoblot assay.

For example, the heterophile test can be used for the diagnosis of infectious mononucleosis (IM) in children and adults. In the test for this antibody, human serum is absorbed with guinea pig kidney, and the heterophile titer is defined as the greatest serum dilution that agglutinates sheep, horse, or cow erythrocytes. Although heterophile antibody binds to certain animal erythrocytes, it does not interact with EBV proteins. A titer of ≧40-fold is diagnostic of acute EBV infection in a patient who has symptoms compatible with 1M and atypical lymphocytes. Tests for heterophile antibodies are positive in 40% of patients with 1M during the first week of illness and in 80-90% during the third week. Tests usually remain positive for 3 months after the onset of illness, but heterophile antibodies can persist for up to 1 year. These antibodies usually are not detectable in children <5 years of age, in the elderly, or in patients presenting with symptoms not typical of IM. The commercially available monospot test for heterophile antibodies is somewhat more sensitive than the classic heterophile test. The monospot test is ˜75% sensitive and ˜90% specific compared with EBV-specific serologies. False-positive monospot results are more common among persons with connective tissue disease, lymphoma, viral hepatitis, and malaria.

EBV-specific antibody testing is used for patients with suspected acute EBV infection who lack heterophile antibodies and for patients with atypical infections. Titers of IgM and IgG antibodies to viral capsid antigen (VCA) are elevated in the serum of more than 90% of patients at the onset of disease. IgM antibody to VCA is most useful for the diagnosis of acute EBV infection because it is present at elevated titers only during the first 2-3 months of the disease. In contrast, IgG antibody to VCA is usually not useful for diagnosis of EBV infection but is often used to assess past exposure to EBV because it persists for life.

Seroconversion to EBNA positivity is also useful for the diagnosis of acute infection with EBV. Antibodies to EBNA become detectable relatively late (3-6 weeks after the onset of symptoms) in nearly all cases of acute EBV infection and persist for the lifetime of the patient. These antibodies may be lacking in immunodeficient patients and in those with chronic active EBV infection.

Titers of other antibodies may also be elevated in patients with EBV infection. Antibodies to early antigens (EAs) are found either in a diffuse pattern in the nucleus and cytoplasm of infected cells (EA-D antibody) or restricted to the cytoplasm (EA-R antibody). These antibodies are detectable 3-4 weeks after the onset of symptoms in patients with EBV infection. About 70% of individuals with EBV infection have EA-D antibodies during the illness. The presence of EA-D antibodies is especially likely in patients with relatively severe disease. These antibodies usually persist for only 3-6 months. Levels of EA-D antibodies are also elevated in patients with nasopharyngeal carcinoma or chronic active EBV infection. EA-R antibodies are only occasionally detected in patients with EBV infection but are often found at elevated titers in patients with African Burkitt's lymphoma or chronic active EBV infection. IgA antibodies to EBV antigens have proved useful for the identification of patients with nasopharyngeal carcinoma and of persons at high risk for the disease.

EBV infection can also be detected using, e.g., real-time PCR assays (see, e.g., Kimura et al., J. Clin. Microbiol. 37:132-136, 1999; Pitetti et al., Pediatr. Infect. Dis. J. 22:736-739, 2003; and Bauer et al., J. Med. Virol. 75:54-58, 2005).

The following examples are meant to illustrate the invention. They are not meant to limit the invention in any way.

### EXAMPLES

### Example 1

### Phase I Clinical Trial for the Treatment of Type I Diabetes

A phase I clinical trial in humans with established type 1 diabetes was performed. The double-double blinded and placebo-controlled study used two separate vaccinations with very low dose BCG spaced four weeks apart. The subjects were studied using intense blood monitoring of the number of autoreactive T cells and the number of T regulatory cells, and the pancreas was also monitored for signs of regeneration. Although almost all immunomodulatory trials in type 1 diabetes are conducted in very-recent-onset diabetics with the aim of slowing the inevitable decline in the pancreas function with immunosuppression, the study was conducted in subjects with longstanding type 1 diabetes, on average 15 years duration, having no clinically-detectable pancreas function.

### Methods

### Study Design

This clinical trial, using type 1 diabetics with long standing disease, was performed to investigate whether BCG administration would result in an immunomodulatory or pancreas inductive effect. In the trial, one group of diabetics was vaccinated with BCG (n=3) and one group with saline (n=3). One saline-injected placebo-treated diabetic was experiencing an acute systemic EBV infection, which was simultaneously followed, as another pathogen like BCG, that induces TNF. The BCG vaccination was administered intradermally on two separate occasions, with a four-week interval between the two injections. Blood was drawn from six normal control volunteers who were studied simultaneously with each diabetic sample. Both the diabetic blood and control blood was blinded and simultaneously sent to the laboratory for monitoring of the T cell response. Serum was saved for the monitoring of pancreas function (autoantibody and C-peptide) and sent to outside vendors for analysis.

Diabetics for this study were people with established disease and ranged in age from 18 to 50 years of age with no demonstrable insulin secretion by a standard C peptide assay. The diabetic inclusion criteria were type 1 diabetics treated continuously with insulin from the time of diagnosis, age 18 to 50, anti-GAD positive, HIV antibody negative, normal complete blood count (CBC), negative purified protein derivative (PPD), and human chorionic gonadotropin (HCG) negative. Exclusion criteria included diabetics with chronic infectious disease such as HIV, history of tuberculosis, current treatment with glucocorticoids, history of HIV, chronic immunosuppressive medications, or high dose aspirin (>160 mg/day). Diabetics with keloid formation, HbA1Cs of greater than 8%, pregnant or not using acceptable birth control or living with someone who is immunosuppressed were also excluded from the study. For the control non-diabetic subjects, inclusion criteria were an age of 18-45, no history of autoimmune disease or diabetes, no history of HIV, and no history of autoimmunity in first-degree family members.

If successful enrollment occurred, whether a diabetic subject (n=6) or the control subject (n=6), all subjects were studied weekly from baseline (week 0) to week 8, bi-weekly during weeks 10 and 12, and a final study time at week 20. Subject participation in the study spanned an approximate 5-month period. Each diabetic volunteer was paired with a non-diabetic control (n=6) that was subject to identical blood draws for monitoring the immune response, i.e., all blinded blood samples were sent to the laboratory for testing of T cell or serum immune responses. All blood was used fresh and processed within two hours of the blood draw. Control subjects undergoing the frequent blood draws were not vaccinated with BCG. Each patient (treated or untreated) and non-diabetic control set were labeled Cambridge, Jefferson, Lincoln, Melrose, Nantucket, or Andover throughout the study.

The first two BCG vaccinations or saline administrations were performed in the clinic using a randomization scheme prepared in the research pharmacy to either administer BCG or saline vaccination. BCG vaccination was performed with the standard method of intradermal injections of 0.1 mL containing 1.6-3.2×10⁶ cfu per injection (prepared from lyophilized BCG, Theracyc, Sanofi-Pasteur, Toronto, Ontario, Canada) into the deltoid area. Placebo controls were injected at the same site with a similar volume of saline. BCG is made from the Mycobacterium Bovis baccilus Calmette-Guerin (BCG), an avirulent strain different from the strain causing tuberculosis in humans, i.e., Mycobacterium tuberculosis. The syringes were prefilled in the research pharmacy. The study staff administered vaccine or placebo, and were not the same staff used to examine the subject to grade the vaccination site. A repeat vaccination with BCG in the same volume again prepared by the Research Pharmacy was performed four weeks after the first immunization.

### Immune Monitoring

### T Cell Assays

CD4⁺ and CD8⁺ T cells were isolated from fresh human blood within 1.5 h of venipuncture using Dynal CD4 positive isolation kit and Dynal CD8 positive isolation kit (Invitrogen, Carlsbad, Calif.). This method is unique in yielding cells that are both free of magnetic particles and free of the positive selection with the antibody. The blood was drawn into BD Vacutainer tubes (BD). The CD8⁺ and CD4⁺ cells used for these studies were prepared from fresh blood, and had to have greater than 98% purity, 95% viability, and 85% yield for the following assays to be standardized.

### Detection of Autoreactive CD8⁺ T Cells in Type 1 Diabetes

Highly purified, viable and high yield CD8⁺ T cells were utilized for tetramer staining, as previously described (Verginis et al., Proc. Natl. Acad. Sci. U.S.A. 105:3479-3484, 2008). Tetramers are diagnostic reagents that are composed of the binding region of specific HLA class I proteins with loaded peptides in the exterior binding groves. The tetramers are then made fluorescent and act as diagnostic reagents that can bind to T cells with specific reactivity to the presented peptide fragment. For detection of autoreactive T cells to insulin, tetramers to HLA *0210 insulin beta 10-18 with a fragment of HLVEALYLV (Beckman Coulter #T02001) were used. For negative control tetramers, the following tetramer reagents were used: HLA *0201 Her-2/neu with a sequence to KIFGSLAFL (Beckman Coulter #T02001), a breast cancer peptide, HLA *0201 null without a non-specific peptide fragment (Beckman Coulter #T01010) and/or a tetramer to the CMV virus HLA-A *0201 CMGPP65 with a sequence of NLVPMVATV (Beckman Coulter #T01009).

Tetramer reagent staining was conducted both after 12 h of culture at 26° C. followed by 6 h at 37° C. and/or after 1 h rest at 26° C. followed by 12 h at 37° C. The cells were then stained with Sytox-green (MBL International Co., Woburn, Mass.) and/or CD8 antibodies (BD Biosciences, San Jose, Calif.). All cells were stained at 4° C. in the dark for 30 minutes and then washed twice in Hank's buffer with 2% heat-inactivated bovine serum. On average, 100,000 highly pure CD8⁺ T cells were analyzed to ensure clear data points on the Becton Dickinson FACSCalibur using the Cell Quest acquisition program and to allow for the detection of the rare autoreactive T cells. All cells were fresh to prevent fixation artifacts and to allow for the quantification of dead versus viable cells. All cells for tetramer staining were never frozen nor expanded before study.

For calculations of insulin tetramer positive cells, the percentage of tetramer insulin or EBV positive cells in total cells was used. For diabetic patients used in the study, all were HLA-A2 positive, except for Melrose. Non-diabetic controls in this study (Jefferson, Lincoln, and Nantucket controls) were HLA-A2⁺. For the flow cytometry studies, the flow gates were set “open” for inclusion of CD8⁺ and CD4⁺ cells of all sizes but excluded cell debris, red blood cells, fragmented cells, and apoptotic bodies. The “open gate” was chosen on purified CDC and CD4⁺ T cells, because T cells undergoing cell death, especially by apoptosis, display changes in light scattering properties. Cell viability was quantified by either of two stains that fluorescently label dead cells, Sytox (MBL International Co., Woburn, Mass.) or propidium iodide (PI).

### Detection of T_{REG} CD4⁺ Cells in Type I Diabetics

Two different methods were used for the detection of T_{REG} cells. For both methods, highly purified fresh CD4⁺ cells were the starting cells for the detection of T_{REG} cells. T_{REG} cells were detected using CD4, CD25^{bright}, and Foxp3 staining or with CD4, CD25^{bright}, and CD127^{low} antibody staining. Intracellular staining of Foxp3 was performed with Human Treg Flow Kit (Biolegend, San Diego, Calif.) according to the manufacturer's instructions. Briefly, isolated CD4 positive cells were incubated with CD4-PE-Cy5 (clone RPA-T4) and CD25-PE (clone BC96) antibodies for 20 minutes at room temperature. After washing, cells were fixed with Foxp3 Fix/Perm solution (Biolegend) for 20 minutes at room temperature. Cells were washed and permeabilized with Biolegend's Foxp3 Perm Buffer for 15 minutes at room temperature. Cells were then stained with Foxp3 Alexa Fluor477 antibody (clone 259D) for 30 minutes. Isotype controls were done for each sample prior to flow cytometric analysis. Alternatively, for detection of T regulatory cells, staining was also performed with a CD4 antibody (clone RPA-T4, BD Biosciences, San Jose, Calif.) and an anti-human CD127 antibody (clone hIL-7R-M21, BD Biosciences).

### Serum Assays

Fresh human blood was collected by venipuncture, allowed to clot, and then serum was separated by centrifugation within 2 h of venipuncture. Serum was stored at −80° C. until analysis.

For serum assays sent out to commercial sources, both published and inter assay viabilities were used for the statistical analysis. Interassay variability was verified for clinical samples by examining the pre-screening values with all baseline values to the published inter assay variability. This analysis was performed for serum assays such as C-peptide assays and the autoantibody assays.

### EBV Assays

EBV was diagnosed in an untreated diabetic patient that presented with severe fatigue at week 2 after the baseline placebo injection and was also observed in the flow cytometric assay for EBV positive CD8⁺ T cells in the laboratory. Further confirmation of this diagnosis was obtained at the end of the trial with serology sent for commercial testing (Quest Diagnostics, Cambridge, Mass.). This long-term diabetic, also known as Andover, was tested for both EBV Early Antigen D antibodies, a sign of an infection that is less than 3 weeks of duration, and less than 2 months in duration, and screened for EBV VCA antibody (IgM), a sign of an infection that is 3-4 weeks in duration.

### Pancreas Monitoring

Two different methods were used to assess whether the subjects had a pancreas response to the BCG vaccination: insulin C-peptide levels and autoantibody levels. For enrollment into this study, a requirement was that each subject have undetectable C-peptide levels by the standard clinical C-peptide assay. Blood for prospective patients was screened for clinically detectable C peptide at the Mayo Clinic and this laboratory utilizes the Cobas C-peptide assay (Roche Diagnostics). This assay has a range of detection of 330-1470 pmol/L for C-peptide. For the monitoring of pancreas function, serum was saved and sent to Sweden for analysis in an ultrasensitive C peptide assay with a lower level of detection of 1.5 pmol/L and an assay range of up to 285 pmol/L for C peptide (Mercodia, Stockholm, Sweden).

Prior to enrollment in this study, a single serum sample for GAD autoantibody from each subject was either sent to the Joslin clinic (Andover, Cambridge, Jefferson, and Melrose) or sent to Quest Diagnostics (Lincoln and Nantucket). For enrollment in this study, patients had to be autoantibody positive. For the baseline through week 20 serum samples after the first BCG or placebo injection, 5-month collected serum samples were sent to Germany for diabetic autoantibody panels, i.e., the laboratories of Drs. Ezio Bonifacio and Peter Achenbach. The autoantibodies studied were anti-IA-2A (anti-protein tyrosine phosphatase), anti-GAD (anti-pancreatic glutamic acid decarboxylase), and anti-ZnTSCarg-A (anti-pancreatic beta cell-specific zinc transporter) (Munich, Germany).

### Statistical Analysis

For statistical comparisons between treatment (BCG-treated or EBV-infected) to placebo subjects (untreated) or BCG-treated to EBV-infected, a paired t-test and Wilcoxon signed rank test were used. For many of the immune assays, i.e., the qualifications of the change in insulin autoreactive T cells, the area under the curve defined as the area of the curve above the simultaneously studied non-diabetic control samples were used. Mean and standard deviation of the quantities were calculated. P-values of <0.05 were considered statistically significant. SAS version P.12 (SAS Institute) was used for the statistical analysis. For autoantibody assays, the known intra assay sensitivity was considered and a comparison to baseline patient values were used to see if a post-baseline change had occurred after treatment. For C-peptide assays both the sensitivity of the assay was considered and compared to either the pre-treatment values or the peak change was compared to other values of that same patient. For example, the p values were derived from hypothesis testing the observations that Cambridge appears to have an elevated C peptide level from weeks 7-12 compared to C peptide levels from pre- to week 5. The hypothesis was tested that Jefferson from week 4, three hours, through week 4, 1 day had elevated C peptide levels compared to either baseline to week 3 or to week 6 to week 10. For Andover, the hypothesis was tested that elevated C peptide occurred from week 4 through week 4, day 1 compared to either pre-week to week 3 or to week 5 to week 12. All C-peptide and autoantibody data is also represented as the probabilities based on a consideration of the inter assay variability for this particular monitored immune parameter. If the serum assays were sent out to commercial sources for evaluation, both published and inter assay variability was used for the statistical analysis. In some data, the pre-screening values to post-baseline values were examined for trends. This analysis was performed for serum assays, such as C-peptide and autoantibodies assays, which were done by outside commercial sources.

### Results

Type 1 diabetic subjects recruited for the trial had long-standing diabetes, but with a persistent measurable autoantibody response to GAD and undetectable pancreatic insulin secretion by a standard clinical assay for C-peptide. All enrolled type 1 diabetic subjects were also required to have a H1A1c of <8%.

Type 1 diabetic subjects enrolled in the trial had an average disease duration of 15 years (range 7-23 years) and an average age of 33 years old (range 26-47) (Table 3). Also, one diabetic at the time of enrollment to the placebo limb of this trial had an undiagnosed case of acute Epstein-Barr virus (EBV) infection that presented clinically as fatigue and malaise at weeks 2-4 of the trial. This patient was followed continuously for the 5-month trial interval and was compared to the BCG-treated and untreated diabetic volunteers.

TABLE 3
Baseline Characteristics of Type I Diabetics

| | | | | Cambridge | Jefferson | Lincoln | Melrose | Nantucket | Andover |
|---|---|---|---|---|---|---|---|---|---|
| | | | Mean | BCG Treated | | | Untreated | | EBV Infected |
| Age | | | 35 | 33 | 28 | 26 | 37 | 41 | 47 |
| Gender (M/F) | | | 5/1 | M | F | M | M | M | M |
| Body Weight (Kg) | | | 84 | 86.4 | 72.7 | 77.2 | 84.9 | 90.18 | 92.8 |
| Duration of Disease | | | 15 | 23 | 7 | 24 | 13 | 8 | 17 |
| Age of Onset of Disease | | | 20 | 10 | 21 | 2 | 24 | 33 | 30 |
| HbA1c | Pre-trial | Week 0 | 6.9 | 7.3 | 6.7 | 5.9 | 7.6 | 7.7 | 6.2 |
| | Mid-trial | Week 5 | 7.05 | 7.9 | 6.8 | 5.9 | 7.6 | 7.5 | 6.6 |
| | Post-trial | Week 12 | 7.12 | 8 | 7.2 | 6.1 | 8 | 7.2 | 6.2 |

| | | | | | | | |
|---|---|---|---|---|---|---|---|
| Serum glucose at Screening | 179 | 169 | 358 | 127 | 199 | 171 | 50 |
| C-peptide at Screening* | 0.23 | 0.1 | 0.1 | 0.1 | 0.6 | 0.4 | 0.1 |

*The screening of C-peptide levels was performed with the standard clinical test (Roche Diagnostics, Cobras, Indianapolis, IN) that typically has a sensitivity range of 370-1470 pmol/L.

### Effect of BCG Vaccination and EBV Infection on Insulin Autoreactive T Cells

Autoreactive T cells in diabetics show cytotoxicity against self-peptides correctly presented through HLA class I alleles. For example, peptide-specific autoreactive T cells against the insulin B chain 10-18, detected in vitro with the insulin tetramer reagent, have been identified in rare islet allograft recipients and in about 20-35% of long-term diabetic patients, when improved blood isolation methods are utilized. Insulin autoreactive T cells detected in blood are rare and found at levels of 0.22-1.5% of the CD8⁺ T cells in long-term diabetic patients (Verginis et al., Proc. Natl. Acad. Sci. U.S.A. 105:3479-3484, 2008). In culture, these insulin autoreactive cells selectively die with short exposures to added TNF. In this study, the effect of endogenous TNF elevations secondary to either BCG vaccinations or an acute EBV infection on in vivo autoreactive T cells was determined.

As a first step in the study, the presence of detectable CD8⁺ T cells with reactivity to the insulin peptide fragment in the HLA-A2 (*0201) allele was measured in each long-term diabetic subject. As the double-blinded placebo-controlled data shows, unmasking of all six diabetic and control blood samples revealed that, at baseline (B), the day of enrollment, none of the six long term diabetics had detectable insulin autoreactive T cells compared to non-diabetic control samples, which were simultaneously studied. This is consistent with the frequency of diabetic patients expressing these rare cells with the established disease.

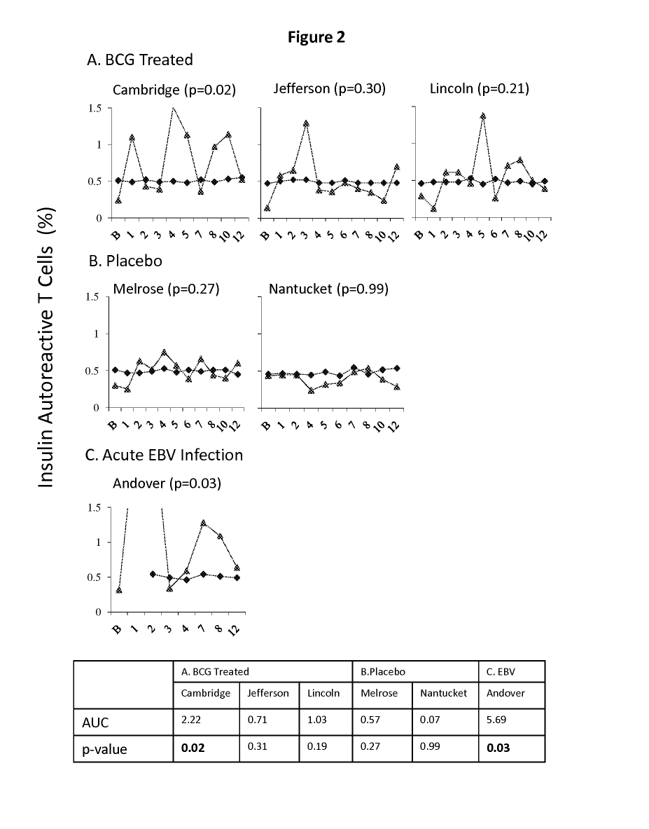

Analyzing all diabetic subjects and matched non-diabetic controls at frequent weekly or bi-weekly intervals over the course of this study revealed new patterns of circulating autoreactive T cells to the insulin peptide fragment. Generally, within about 1-3 weeks of the first treatment, either after BCG treatment or with an acute ongoing EBV viral infection, detectable numbers of insulin-specific autoreactive T cells appeared in the circulation (FIGS. 2A and 2C). The two placebo-treated diabetic patients showed little if any appearance of insulin autoreactive T cells or fluctuations in the numbers of insulin autoreactive cells with sequential blood samples compared to the control samples. The area under the curve (AUC) was calculated for the increased insulin autoreactive T cells for each patient compared to their simultaneous matched control blood samples over the many weeks of monitoring. For the BCG-treated subjects, the area under the curve was 2.22, 0.71, and 1.03, for the EBV-infected subject, the area under the curve was 5.69, and for the two untreated placebo diabetics the area under the curve for insulin autoreactive T cells was 0.57 and 0.07. As is evident from these AUC calculations, the greatest numbers of released insulin autoreactive T cells were during the active EBV infection (p=0.03), but a BCG treated patient (Cambridge) released statistically significant elevated insulin autoreactive T cells after treatment (p=0.02, Cambridge). Both placebo diabetics receiving saline injections did not release insulin autoreactive T cells during the long course of study (p=0.27 and p=0.99).

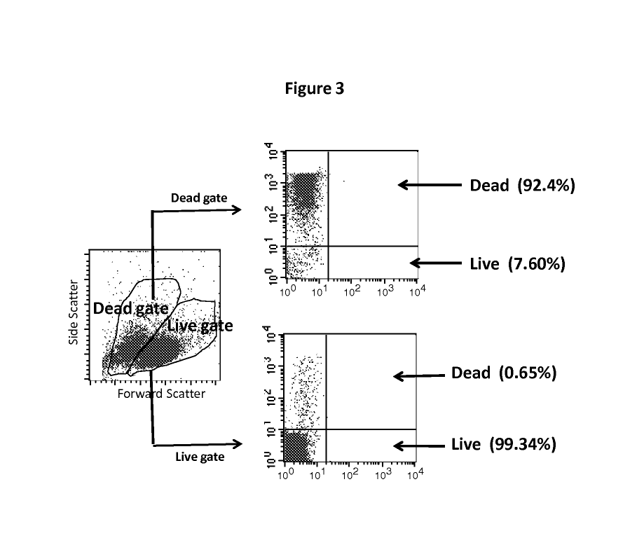

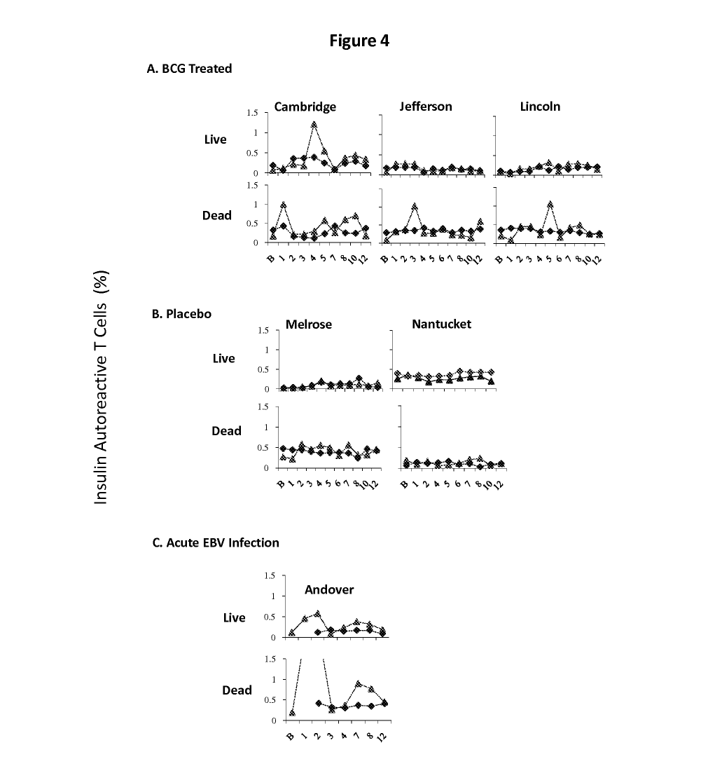

All insulin autoreactive T cells were serially studied for detection in peripheral blood and were also studied for cell viability at each monitoring point (FIG. 3 demonstrates the assay for the measurement of dead and viable CD8⁺ T cells). Increased levels of circulating and released autoreactive CD8⁺ T cells might be predicted to represent dying cells from the boost in TNF levels from either a BCG or EBV infection. Overall, the augmented and detectable insulin autoreactive T cells in the circulation were predominantly dead insulin autoreactive T cells (FIGS. 4A-4C).

### Effect of BCG Vaccinations and EBV Infection on Induction of T_{REG} Cells

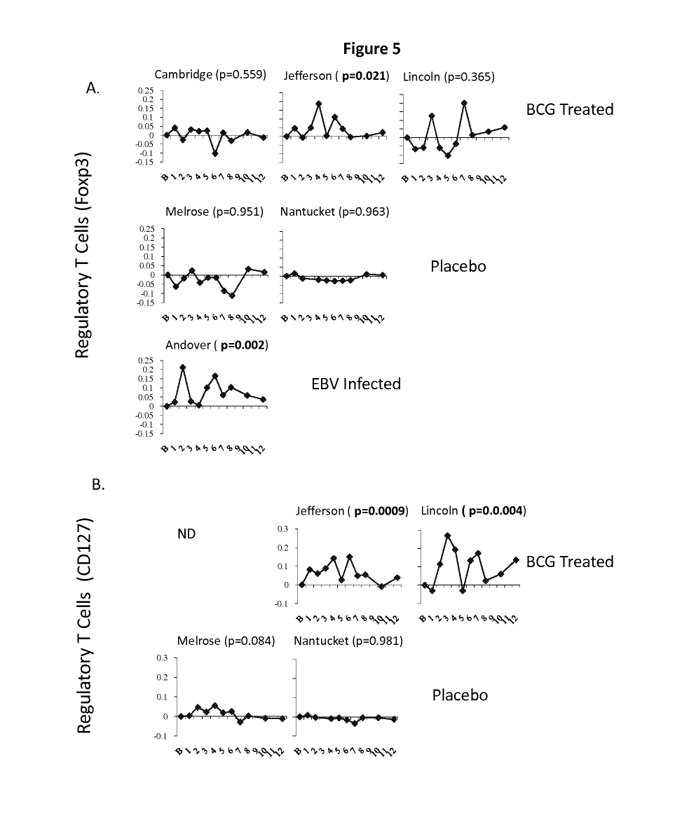

The levels of T_{REG} CD4⁺ T cells were measured in fresh blood specimens from enrollment day (Baseline) till the end of the study at weekly or bi-weekly intervals for all patients (n=6) compared to their simultaneously studied non-diabetic control samples (n=6). Two different T_{REG} analysis methods were used to quantify the possible change in T_{REG} cells with BCG or EBV exposures compared to the placebo diabetics, i.e., the numbers of CD4⁺, CD25⁺, and Foxp3⁺ T cells or the numbers of CD4⁺, CD25⁺, and CD127^{low} T cells. The later method was used if sufficient CD4⁺ T cells were available at any monitoring time. FIG. 5A measured the relative changes in T_{REG} cells co-expressing Foxp3. Using this analysis method, one BCG-treated diabetic (Jefferson) and the EBV-infected diabetic demonstrated statistically significant elevations in the relative numbers of T_{REG} cells (p=0.021 and p=0.002, respectively). The placebo-treated long-term diabetics had no change in the numbers of T_{REG} cells measured by Foxp3 staining (p=0.951 and p=0.963) (FIG. 5A). Using the CD127^{low} method of detecting T_{REG} cells, two of the two BCG-treated diabetics monitored by this method (Jefferson and Lincoln) demonstrated marked inductions of T_{REG} cells (p=0.0009 and p=0.004) and none of the placebo diabetics (Melrose and Nantucket) demonstrated any induction of T_{REG} cells by either method (p=0.084 and p=0.981) (FIG. 5B). Therefore, all BCG-treated and EBV-exposed diabetics, except Cambridge, demonstrated T_{REG} cell induction during the monitoring period of this trial.

### Effect of BCG Vaccination and EBV Infection in Autoantibody Production

Glutamic acid decarboxylase (GAD) is a key autoantigen in type I diabetes and represents a protein specific to the insulin secreting beta cells of the pancreas. The exposure of the immune system to new beta cells of the islets, especially islets from a pancreas or islet cell transplant solicits a rapid change of GAD autoantibodies. Autoantibodies after new islet exposures can fluctuate either upwards or downwards. All diabetic subjects in the trial were monitored for changing GAD autoantibodies. This immune parameter was used as an indirect indication that the pancreas has regenerated.

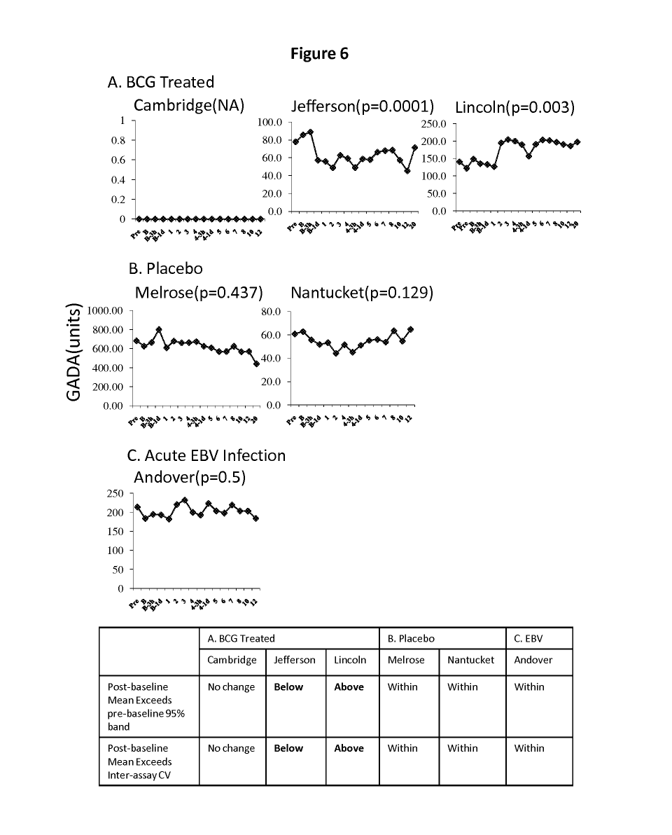

As is typical, the long-term type 1 diabetics, at baseline, had varying amounts of autoantibodies to GAD. At baseline, GAD autoantibodies were detectable in Andover, Jefferson, Melrose, Lincoln, and Nantucket. Both GAD autoantibody positive BCG-treated subjects, Jefferson and Lincoln, demonstrated a rapid change in GAD levels after one week of the first BCG injection (p=0.0001 and p=0.003) (FIG. 6A). The Jefferson diabetic had a statistically significant and large fall in GAD autoantibody levels and the Lincoln diabetic showed a reciprocal rise in GAD autoantibodies (FIG. 6A). The dramatic changes in GAD autoantibodies were not only observed in an analysis of pre-GAD levels to post-treatment levels, but also the limits of the assay were considered to see if these changes could be verified based on the post-baseline mean exceeding the inter-assay CV. The bottom of FIG. 6 shows that the same trends were observed with these alternate methods of analysis.

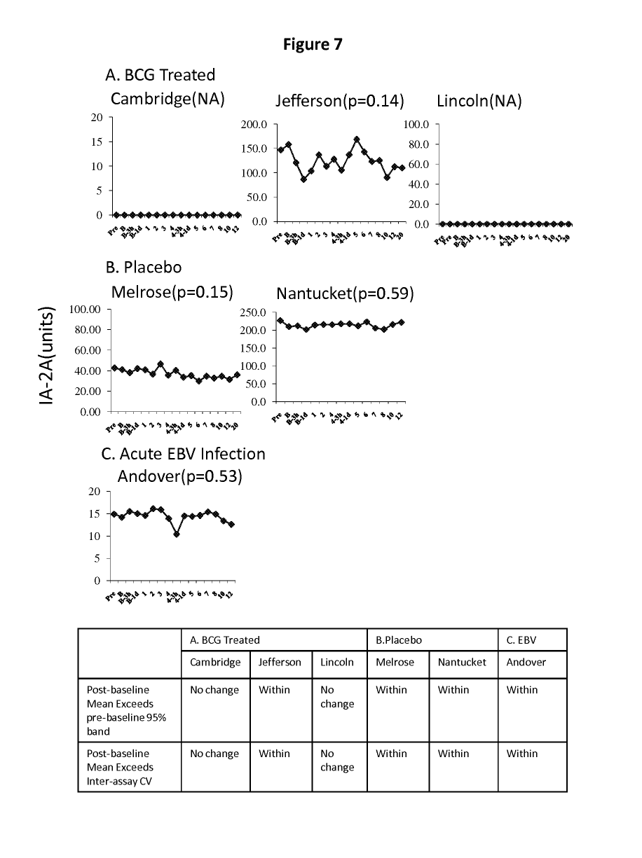

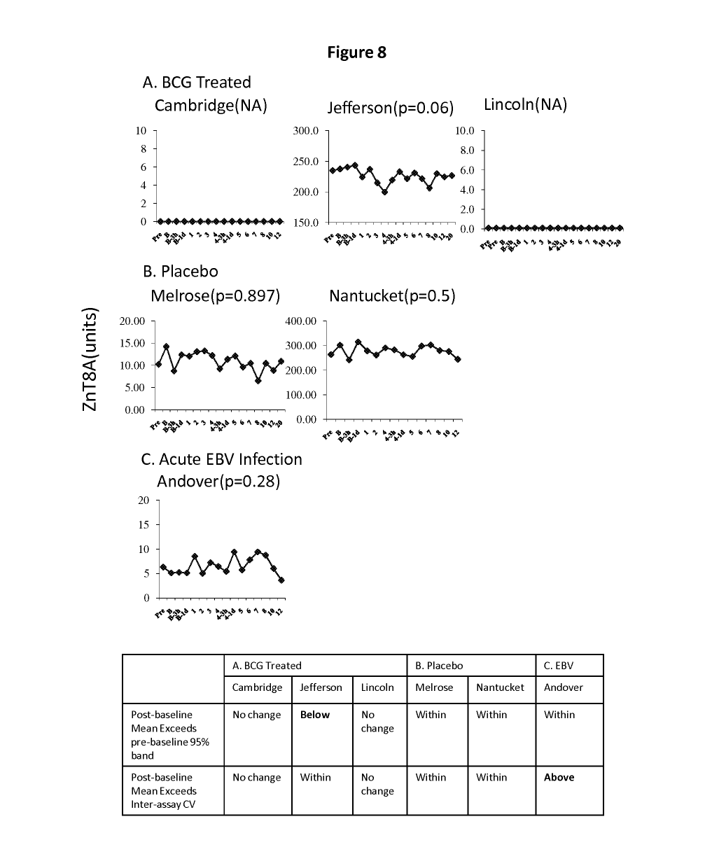

In addition to monitoring GAD autoantibodies, all patients were also monitored for protein tyrosine phosphatase (IA-2A) and pancreatic beta cell-specific zinc transporter (ZnT8A) autoantibodies. There was no statistically significant change in IA-2A autoantibodies in any of the diabetic subjects from any of the groups (FIGS. 7A-7C). Only ZnT8A autoantibodies, as indicated by one statistical method in Jefferson, demonstrated a fall in levels (FIGS. 8A-8C), which is a trend similar to the fall in GAD autoantibodies also observed in the same subject (FIG. 6, bottom).

### Effect of BCG Vaccination and EBV Infection on Insulin Secretion

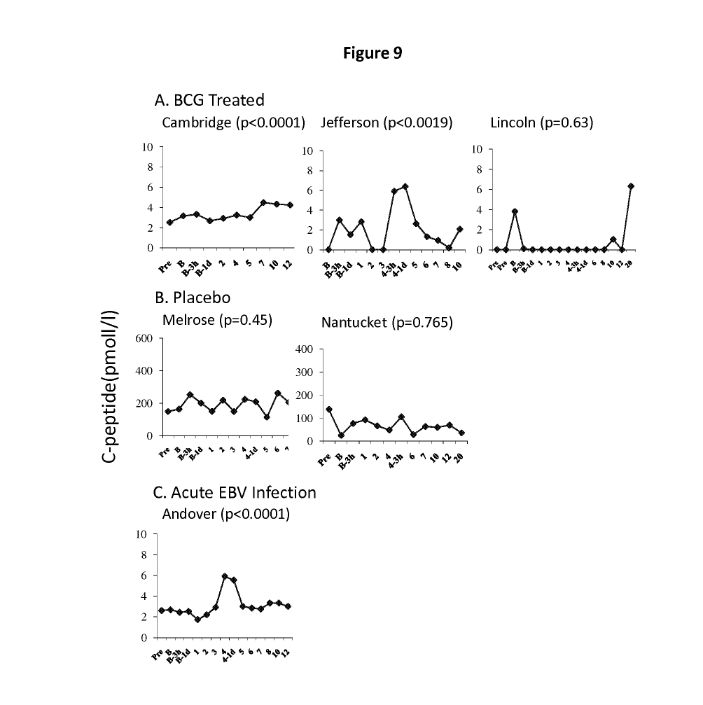

The secretion of pancreatic insulin is associated with the co-secretion of the pro-insulin fragment, i.e., the C-peptide fragment. Throughout the course of the study, all diabetics were monitored weekly or bi-weekly for changes in fasting C-peptide levels, a marker of restored endogenous pancreatic islet activity. To see if C-peptide changed a hypothesis was formulated that C-peptide levels had changed during certain intervals and this data was compared to the baseline values. As FIGS. 9A-9C show, two of the three BCG-treated subjects, Cambridge and Jefferson, who at enrollment had no detectable C-peptide (even in the extremely sensitive C-peptide assay) had a statistically significant change upward in C-peptide levels. For Cambridge, the undetectable C-peptide levels started to rise at week 7 and continued through the last monitoring time (p<0.0001) (FIG. 9A). For Jefferson, the rise in C-peptide only occurred briefly during the week 4-5 intervals (p<0.0019) (FIG. 9A). Similar to Jefferson, the EBV-infected subject, Andover, also briefly demonstrated a statistically significant increase in C-peptide level during weeks 4-5 (p<0.0001) (FIG. 9C). The BCG-treated diabetes subject Jefferson had no change in C-peptide (p=0.63) and both the placebo-treated diabetes subjects, Melrose and Nantucket, had no statistical change in fasting C-peptide levels during the study (FIG. 9B).

### Diagnosis of EBV Infection

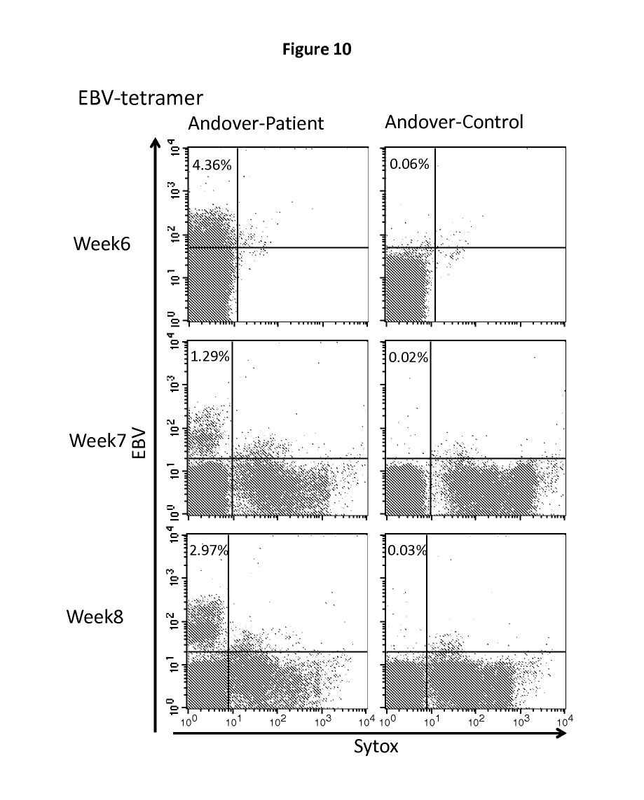

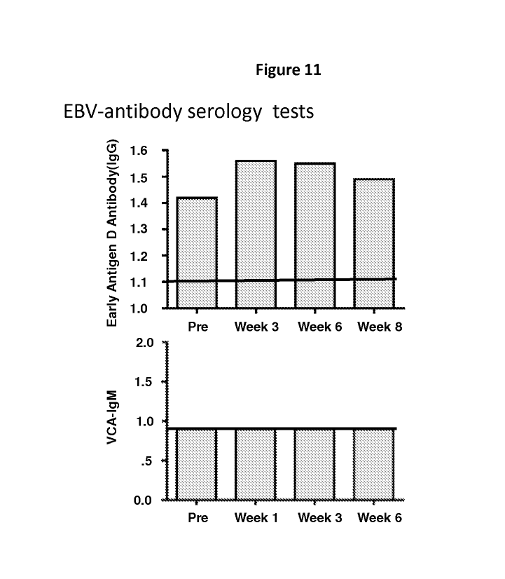

The time course of acute EBV infection in the diabetic patient within the Andover patient group was accurately mapped using multiple methods. First, a control EBV detection reagent was used in parallel with the detection of insulin autoreactive T cells, i.e., the EBV tetramer reagent. The EBV detection reagent became vividly positive between weeks 6-8 in the diabetic subject Andover (Andover-Patient) compared to the paired non-diabetic control subject (Andover-Control) (FIG. 10). Second, subject serum samples were sent for the detection of both early EBV infection (VCA-IgM, positive from day 4 of the injection to week 3) and ongoing EBV infection (Early Antigen D IgG from day 30 to one month of infection). The data show that the EBV infection in the Andover subject was about 30 days duration at the time of enrollment in the trial (FIG. 11).

### Effect of BCG Vaccination and EBV Infection on Routine Chemistries and HbA1c

For all diabetics in the trial, numerous routine chemistries were studied with no changes in the white blood cell, platelet, granulocytes, or hematocrit values. There was also no statistical change in the HbA1c of the diabetic subjects throughout the trial.

### Safety Tests

All patients receiving the generic BCG vaccine developed injection site inflammation at 2-4 weeks. The observed site inflammation healed over weeks to form the typical small circulate scar of the BCG vaccine (REF). All patients were without reportable systemic symptoms other than the upper arm scar, except for the untreated diabetic that presented with an acute EBV infection, who had symptoms of fatigue and malaise.

These data indicate that vaccination with a virus (e.g., EBV) or treatment with a viral polypeptide or a nucleic acid the encodes a polypeptide that induces TNF production or activates a TNF signaling pathway in a subject having an autoimmune disease may provide treatment of the autoimmune disease (e.g., a release of dead autoreactive CD8⁺ T cells, an increase in T_{REG} cells, and/or an increase in C-peptide levels).

### Example 2

### In Vitro Assays of CD8⁺ T Cell Death

Assays to determine the ability of the compositions of the invention to mediate autoreactive CD8⁺ T cell death may be performed using the in vitro assay described in Ban et al. (Proc. Natl. Acad. Sci. U.S.A. 105:13644-13649, 2008). In an example of this assay, viable subpopulations of CD4⁺ and CD8⁺ T cells may be isolated from an autoimmune patient (e.g., a type I diabetic subject) and incubated with a composition of the invention. The ability of the composition to elicit cell death in the CD4⁺ and CD8⁺ populations may be measured using two different cell death assays: lactate dehydrogenase (LDII) assay (necrotic cell death) and the caspase 3/7 assay (a luminescent assay of apoptosis).

Similar experiments to determine the effect of a composition of the invention on cell death in autoreactive diabetic CD8⁺ T cells to an insulin fragment may also be performed. These assays are performed by first incubating purified CD8⁺ T cells with fluorescently-labeled tetramers to HLA 0210 with an insulin beta 10-18 fragment (HLVEALYLV), washing the cells twice in Hank's buffer with 2% heat inactivated bovine serum, and isolating the labeled autoreactive CD8⁺ T cells using a flow cytometer. The isolated autoreactive CD8⁺ T cells are then treated with compositions of the invention and the amount of cell death (necrotic and/or apoptotic cell death) is measured using either a LDH assay or a caspase 3/7 assay (described above). Compositions useful for treatment will result in an increase in necrotic or apoptotic autoreactive CD8⁺ T cell death. Suitable controls for these experiments will include CD8⁺ T cells from a subject that does not have insulin-dependent diabetes, samples of untreated autoreactive CD8⁺ T cells, or samples of autoreactive CDC T cells treated with a control polypeptide (e.g., albumin).

### Example 3

### Mouse Model of Insulin-Dependent Diabetes

A mouse model of insulin-dependent diabetes (NOD mice) may be used to determine whether the dosing and efficacy of a composition of the invention for treatment of insulin-dependent diabetes. The NOD mice can be administered a composition containing live EBV, live attenuated EBV, dead (e.g., heat-inactivated) EBV, or any of the proteins of EBV that induce an elevation in the level of TNF-α (e.g., the concentration of TNF-α found in the blood) (e.g., LMP1). The mice will subsequently be assessed for a change in the symptoms of insulin-dependent diabetes: thirst, frequent urination, extreme hunger, weight loss, fatigue, blurred vision, and ketoacidosis (DKA). In addition, the mice will be monitored for an increase in pancreatic β-cell number, an increase in insulin production, an increase in C-peptide levels, a decrease in glycated hemoglobin A1c levels, an increase in autoreactive CD8⁺ T cell death (e.g., apoptosis), or increase in the number of regulatory T cells. The mice will also be monitored for a decrease in the levels of autoantibodies, such as anti-glutamic acid dehydrogenase (anti-GAD) auto-antibodies and anti-pancreatic beta cell-specific zinc transporter (anti-ZnT8A) auto-antibodies. The experimental values for the treated mice will be compared relative to control mice (e.g., a NOD mouse not receiving the composition or receiving a placebo, or a biological sample from the NOD mouse prior to treatment with the composition). A composition that may be used to treat a subject having insulin-dependent diabetes will desirably result in a decrease or alleviation of one or more symptoms of insulin-dependent diabetes, an increase in pancreatic β-cell number, an increase in insulin production, an increase in C-peptide levels, a decrease in glycated hemoglobin A1c levels, an increase in autoreactive CD8⁺ T cell death (e.g., apoptosis), or an increase in the number of regulatory T cells in the treated mice compared to the control mice.

### Example 4

### Rat Model of Insulin-Dependent Diabetes

A rat model of insulin-dependent diabetes (BB rats or diabetes-prone DP-BB/Wor rats) may be used to determine whether the dosing and efficacy of a composition of the invention for treatment of insulin-dependent diabetes (Whalen et al., Current Protocols Immunology, Chapter 15, 2001). The BB rats can be administered a composition containing live EBV, live attenuated EBV, dead (e.g., heat-inactivated) EBV, or any of the proteins of EBV that induce an elevation in the level of TNF-α (e.g., the concentration of TNF-α found in blood) (e.g., LMP1). The rats will subsequently be assessed for a change in the symptoms of insulin-dependent diabetes: thirst, frequent urination, extreme hunger, weight loss, fatigue, blurred vision, and ketoacidosis (DKA). In addition, the rats will be monitored for an increase in pancreatic β-cell number, an increase in insulin production, an increase in C-peptide levels, a decrease in glycated hemoglobin Ale levels, an increase in autoreactive CD8⁺ T cell death (e.g., apoptosis), or increase in the number of regulatory T cells. The rats will also be monitored for a decrease in the levels of autoantibodies, such as anti-glutamic acid dehydrogenase (anti-GAD) auto-antibodies and anti-pancreatic beta cell-specific zinc transporter (anti-ZnT8A) auto-antibodies. The experimental values for the treated rats will be compared relative to control rats (e.g., a BB rat not receiving the composition or receiving a placebo, or a biological sample from the BB rat prior to treatment with the composition). A composition that may be used to treat a subject having insulin-dependent diabetes will desirably result in a decrease or alleviation of one or more symptoms of insulin-dependent diabetes, an increase in pancreatic β-cell number, an increase in insulin production, an increase in C-peptide levels, a decrease in glycated hemoglobin A1c levels, an increase in autoreactive CD8⁺ T cell death (e.g., apoptosis), or an increase in the number of regulatory T cells in the treated rats compared to the control rats.

### Example 5

### Mouse Model of Lupus

A mouse model of lupus ((NZB×NZW)F1, hereafter “NZB mice,” or MRL/lpr mice) may be used to determine whether a composition will provide effective treatment for lupus (Thacker et al., Lupus 19:288-299, 2010). NZB mice spontaneously develop an autoimmune syndrome with notable similarities to human systemic lupus erythematosus. In these experiments, NZB mice can be administered a composition containing live EBV, live attenuated EBV, dead (e.g., heat-inactivated) EBV, or any of the proteins of EBV that induce TNF levels an elevation in the level of TNF-α (e.g., the concentration of TNF-α found in blood) (e.g., LMP1). In these experiments, the NZB mice may be administered a composition of the invention after the development of lupus symptoms. The mice will subsequently be assessed for a change in the symptoms of lupus, for example, the treated mice will be observed for a decrease in the production of IgG antinuclear antibodies, decreased proteinuria, decreased mesangial matrix deposition, decreased anemia, decreased leukopenia, decreased thrombocytopenia, an increase in autoreactive CD8⁺ T cell death (e.g., apoptosis), or increase in the number of regulatory T cells. The experimental values for the treated mice will be compared relative to control mice (e.g., a mice not receiving the composition or receiving a placebo). A composition that may be used to treat a subject having lupus will desirably result in a decrease or alleviation of one or more symptoms of lupus.

### Example 6

### Mouse Model of Experimental Autoimmune Thyroiditis

A mouse model of experimental autoimmune thyroiditis (EAT) may be used to determine whether a composition will provide effective treatment for Hashimoto's disease. Autoimmune thyroiditis in these mice may be induced by immunization with mouse thyroglobulin (MTg) (Tomazic et al., Clin. Exp. Immunol. 58:83-89, 1984). In these experiments, the mice will be administered a composition of the invention prior to or following administration of MTg (as described in the above administration schedules). The mice will subsequently be assessed for a change in the symptoms of autoimmune thyroiditis, for example, increased production of anti-thyroglobulin autoantibodies and increased lymphoid infiltration of the thyroid gland. The experimental values for the treated mice will be compared relative to control mice (e.g., a MTg-injected mice not receiving the composition or receiving a placebo, or a biological sample from the MTg-injected mouse prior to treatment with the composition). A composition that may be used to treat a subject having Hashimoto's disease will desirably result in a decrease or alleviation of one or more symptoms of Hashimoto's disease, for example, increased production of anti-thyroglobulin autoantibodies, increased lymphoid infiltration of the thyroid gland, fatigue, weight gain, cold intolerance, excessive sleepiness, constipation, and muscle cramps, or an increase in the number of regulatory T cells in the treated mice compared to the control mice.

### Example 7

### Re-Activation of a Latent Endogenous EBV Infection Induces TNF-Alpha

Published data by Cernea et al., Clinical Immunology 134:121, 2010, is now re-analyzed to show the relationship between patients who were previously infected with EBV and anti-CD3 efficacy.

Peripheral blood mononuclear cells (PBMC) were isolated from 82 subjects (31 control subjects (blood donors) and 51 patients with up to 1 year duration of type 1 diabetes mellitus (T1DM)). The median duration was 2.5 months (range: 0.5-12 months). All subjects were positive for at least one biochemical autoantibody (anti-GAD65, anti-ICA512, or anti-insulin if within the first 10 days after commencing insulin therapy). PBMCs were frozen for studies at later times or immediately used for analysis. Amongst these subjects were 6 who received a 14-day course of anti-CD3 mAb as part of segment 1 of an open labeled clinical trial (Protégé, Clinicaltrials.gov NCT00385697). The mean age of these subjects was 26.6±5.9 years (range: 19-34 years) and the mean duration of diabetes was 2.4±0.9 month (range 1-3.5 months). PBMCs from these subjects were isolated before (day 0) and after treatment (days 14, 28, 91, 182, 210, 273) with anti-CD3 mAb, Teplizumab (day 0: 51 μg/m², day 1: 102 μg/m², day 3: 204 μg/m², day 4: 408 μg/m², days 5-13: 816 μg/m²). Samples were also obtained before and at similar time points in three untreated subjects with T1DM enrolled in clinical trials.

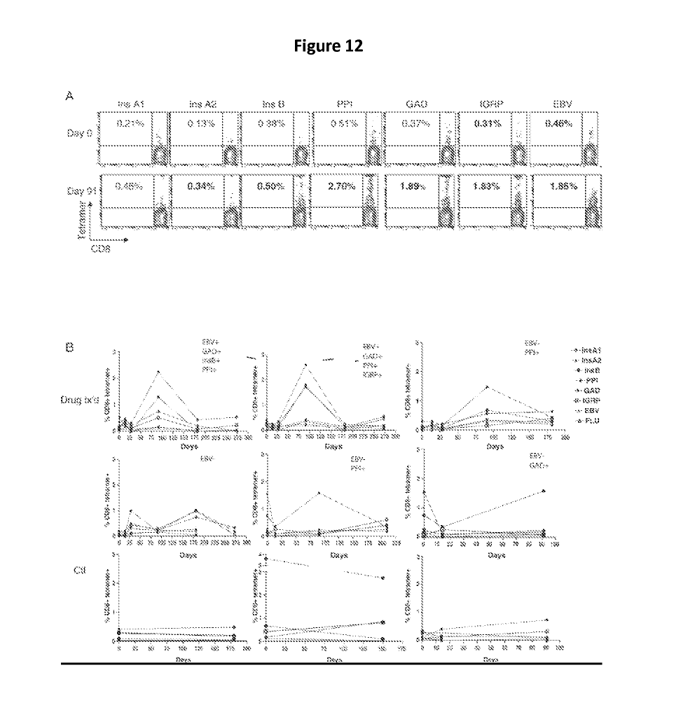

FIG. 12A depicts histograms showing the presence of newly appearing autoreactive T cells from a single participant 3 months (day 90) after treatment with Teplizumab. Although this patient was almost entirely negative for any form of insulin autoreactive T cells after 91 days of therapy, the patient became positive for PPI, GAD and IGRP autoreactive T cells. The newly appearing autoreactive T cells in the circulation correlated with the newly appearing EBV-activated T cells (i.e., from 0.46% to 1.86%). As shown in FIG. 12B, patients 1-4 have newly appearing tetramer+EBV cells and these patients all have the diverse insulin autoreactive T cells. The appearance of diverse autoreactive T cells coincides with the simultaneous detection of EBV subclinical infection. Accordingly, patients 5 and 6 do not have EBV positive cells detectable with EBV and have minimal release of autoreactive T cells (FIG. 12B).

In summary, EBV+ T cells appear with newly appearing autoreactive T cells and the release of diverse autoreactive T cells. Because EBV reactivation occurs at a similar time to the appearance/release of damaged autoreactive cells that were killed, the kinetics of the response indicate a correlation between subclinical EBV and the release of TNF. EBV negative patients do not show the appearance/release of damaged autoreactive cells since there is no EBV to trigger TNF.

The literature shows that for over 15 years infections with EBV are associated with a host response of TNF secretion, a response called innate immunity (see, e.g., Rahman et al., PLoS Pathogens 2:66-77, 2006). Taken together, these data indicate that subclinical EBV releases TNF. Therefore, the dosing of anti-CD3 should be in a therapeutic range which will overlap with detection of EBV or applied to patients who were previously infected with EBV.

### Other Embodiments

All publications and patents cited in this specification are incorporated herein by reference as if each individual publication or patent were specifically and individually indicated to be incorporated by reference, including U.S. Patent Application No. 61/322,695. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

Other embodiments are in the claims.

What is claimed is:

1. A method of treating a mammal having an autoimmune disease comprising administering to the mammal a composition comprising a live, killed, attenuated, or inactivated Epstein Barr Virus (EBV) or all or a portion of at least one EBV polypeptide, or nucleic acid molecule encoding the EBV polypeptide.

2.-5. (canceled)

6. The method of claim 1, wherein said composition comprises two or more EBV polypeptides, or nucleic acid molecules encoding said two or more EBV polypeptides.

7. The method of claim 1, wherein said EBV polypeptide is LMP1.

8. (canceled)

9. The method of claim 1, wherein said composition induces expression of tumor necrosis factor-alpha (TNF-α) in said mammal.

10. (canceled)

11. The method of claim 1, wherein said composition induces activation of the NF-kappa B pathway in an autoreactive immune cell of said mammal.

12. The method of claim 1, wherein said mammal is a human.

13. The method of claim 1, wherein said autoimmune disease is selected from alopecia areata, ankylosing spondylitis, antiphospholipid syndrome, autoimmune Addison's disease, autoimmune hemolytic anemia, autoimmune hepatitis, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatricial pemphigoid, CREST syndrome, cold agglutinin disease, Crohn's disease, discoid lupus, systemic lupus erythmatosous, ulcerative colitis, psoriatic arthritis, essential mixed cryoglobulinemia, fibromyalgia-fibromyositis, Graves' disease, Guillain-Barré, Hashimoto's thyroiditis, hypothyroidism, idiopathic pulmonary fibrosis, idiopathic thrombocytopenia purpura (ITP), IgA nephropathy, insulin dependent diabetes, juvenile arthritis, lichen planus, lupus, Ménière's disease, mixed connective tissue disease, multiple sclerosis, myasthenia gravis, pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjögren's syndrome, Stiff-Man syndrome, Devic's disease, Takayasu arteritis, temporal arteritis/giant cell arteritis, ulcerative colitis, uveitis, vasculitis, vitiligo, and Wegener's granulomatosis.

14. The method of claim 13, wherein said autoimmune disease is insulin-dependent diabetes and wherein said treating results in at least a 1% increase in C-peptide levels in said mammal relative to the C-peptide levels in said mammal prior to said treatment.

15.-26. (canceled)

27. The method of claim 1, wherein said treating induces at least a 1% increase in autoreactive T cell death in said mammal relative to the level of autoreactive T cell death observed in said mammal prior to said treatment and/or induces at least a 1% increase in the number of regulatory T cells in said mammal relative to the number of regulator T cells present in said mammal prior to said treatment and/or induces at least a 1% decrease in autoantibody levels in said mammal relative to the autoantibody levels in said mammal prior to said treatment and/or results in a decrease in one or more symptoms of said autoimmune disease.

28.-33. (canceled)

34. The method of claim 1, wherein said composition is administered parenterally, topically, intravenously, intra-arterially, intracranially, intradermally, subcutaneously, intramuscularly, intraorbitally, intraventricularly, intraspinally, intraperitoneally, intranasally, or orally.

35. The method of claim 1, wherein said composition is administered in one or more doses.

36.-37. (canceled)

38. A method of treating a human subject having an autoimmune disease, said method comprising the steps of:
(a) detecting Epstein-Barr virus (EBV) in said human subject; and
(b) administering an immunosuppressive therapy to said human subject, thereby treating the autoimmune disease in said human subject.

39. The method of claim 38, wherein step (a) comprises detecting tetramer-positive T cells against EBV cells, EBV-specific antibodies, or EBV deoxyribonucleic acid (DNA) or messenger ribonucleic acid (mRNA) molecules.

40. The method of claim 39, wherein the EBV-specific antibodies are viral capsid antigen (VCA)-IgM, VCA-IgG, D early antigen (EA-D)-IgG, or Epstein Barr nuclear antigen-IgG.

41. The method of claim 38, wherein the immunosuppressive therapy comprises an anti-CD3 antibody.

42. The method of claim 41, wherein the anti-CD3 antibody is teplizumab or otelixizumab.

43. (canceled)

44. The method of claim 41, wherein the anti-CD3 antibody is administered in a dosage range of from about 5 μg to about 200 mg.

45. The method of claim 38, wherein said autoimmune disease is selected from alopecia areata, ankylosing spondylitis, antiphospholipid syndrome, autoimmune Addison's disease, autoimmune hemolytic anemia, autoimmune hepatitis, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatricial pemphigoid, CREST syndrome, cold agglutinin disease, Crohn's disease, discoid lupus, systemic lupus erythmatosous, ulcerative colitis, psoriatic arthritis, essential mixed cryoglobulinemia, fibromyalgia-fibromyositis, Graves' disease, Guillain-Barré, Hashimoto's thyroiditis, hypothyroidism, idiopathic pulmonary fibrosis, idiopathic thrombocytopenia purpura (ITP), IgA nephropathy, insulin dependent diabetes, juvenile arthritis, lichen planus, lupus, Ménière's disease, mixed connective tissue disease, multiple sclerosis, myasthenia gravis, pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjögren's syndrome, Stiff-Man syndrome, Devic's disease, Takayasu arteritis, temporal arteritis/giant cell arteritis, ulcerative colitis, uveitis, vasculitis, vitiligo, and Wegener's granulomatosis.

46.-49. (canceled)

50. The method of claim 38, wherein said EBV in said subject is due to a chronic, acute, or subclinical infection or to reactivation of a latent infection.

51. The method of claim 38, wherein said agent is admixed with a pharmaceutically acceptable carrier, excipient, or salt.

52.-104. (canceled)
